(19) United States
(12) Patent Application Publication
(10) Pub. No.: US 2014/0005620 A1
(43) Pub. Date: Jan. 2, 2014

(54) Biodegradable and Flushable Multi-Layered Film

(71) Applicants: James Hongxue Wang, Appleton, WI (US); Gregory James Wideman, Menasha, WI (US)

(72) Inventors: James Hongxue Wang, Appleton, WI (US); Gregory James Wideman, Menasha, WI (US)

(73) Assignee: KIMBERLY-CLARK WORLDWIDE, INC., Neenah, WI (US)

(21) Appl. No.: 13/534,440

(22) Filed: Jun. 27, 2012

(51) Int. Cl. A61F 13/514; B32B 1/02; B29C 47/06; B32B 5/00

(52) U.S. Cl. 604/364; 428/220; 428/35.2; 264/173.16

(57) ABSTRACT

A biodegradable and flushable film is generally provided. The film can be a multi-layer film including a water-dispersible core layer that comprises a water-soluble polymer; and a water-barrier skin layer positioned adjacent to the water-dispersible core layer. The water-dispersible core layer constitutes from about 50 wt. % to about 99 wt. % of the film, while the water-barrier skin layer constitutes from about 1 wt. % to about 50 wt. % of the film. The biodegradable polymers of the water-barrier skin layer can constitute from about 80 wt. % to 100 wt. % of the polymer content in the water-barrier skin layer, with from about 10 wt. % to about 90 wt. % of the biodegradable polymers being polyalkylene carbonate and from about 10 wt. % to about 90 wt. % of the biodegradable polymers being biodegradable polyesters. The film may be used as a packaging film or as a backsheet of an absorbent article.

### BACKGROUND OF THE INVENTION

Disposable absorbent articles are currently used in many different applications, including diapers and training pants for infants and children, feminine care products such as sanitary napkins, pantiliners, or tampons, adult incontinence products, and health care products such as surgical drapes or wound dressings. The disposable absorbent article usually comprises a topsheet, a backsheet and an absorbent core positioned between the backsheet and the topsheet. Depending on the type of use involved, disposable absorbent articles can be subjected to one or more insults from aqueous liquids such as water, urine, menses or blood. As a result, the backsheet materials of these disposable products are typically made of liquid impermeable materials, such as polypropylene or polyethylene films, which exhibit sufficient strength and handling capability so that the disposable absorbent article retains its integrity during use by the wearer and does not allow leakage of the liquid from the product.

Many disposable absorbent articles can be difficult to dispose of into an aqueous environment. For example, attempts to flush many disposable absorbent articles down the toilet can cause blockage of the toilet or pipes connecting the toilet to the sewage system. In particular, the backsheet materials used in these disposable absorbent articles generally do not dissolve, disintegrate or disperse readily when flushed down a toilet so that the disposable absorbent article cannot be disposed of in this manner. If the backsheet materials are made very thin to reduce the overall bulk of the disposable absorbent article and reduce the likelihood of blockage of the toilet or sewage pipe, it may not exhibit sufficient strength to prevent tearing or ripping as the material is subjected to the stresses of normal use by the wearer. In a number of instances, it would be desirable to be able to flush these disposable absorbent articles down the toilet. These include certain catamenial products, known as labial or interlabial sanitary napkins or pads. Interlabial pads have the potential to provide greater freedom from inconvenience because of their small size and reduced risk of leakage. Indeed, these interlabial pads are small enough to be easily flushed down the toilet, typically without clogging it or the sewage pipes. Even though flushable, such products could put a significant environmental demand on sewage treatment or septic tank systems if they are not readily susceptible to degradation and disintegration after being flushed.

Various attempts have therefore been made to solve this problem. For example, U.S. Pat. No. 6,514,602 to Zhao, et al. describes a water-flushable film that contains a water-impervious biodegradable layer and a water-dispersible layer. The biodegradable layer includes from 65% to 100% of a water-insoluble biodegradable thermoplastic polymer and from 0% to 30% of a water-soluble thermoplastic polymer, and the water-dispersible layer contains from 60% to 100% of a water-soluble thermoplastic polymer and from 0 to 40% of a water-insoluble thermoplastic polymer. In one example, the film contains a 1^{st} layer of 25% Bionolle (polybutylene succinate adiapte copolymer) and 75% PEO; a 2^{nd} layer of 25% Bionolle and 75% PEO; and a 3^{rd} layer of 100% Bionolle. Despite imparting some barrier properties to the film, various problems nevertheless remain with such films. For instance, several of the synthetic biodegradable polymers employed in Zhao, et al. can lead to an undesirable stickiness when dry or wet, as well as relatively poor mechanical properties. The polymers are also expensive. Furthermore, while the synthetic biodegradable polymers employed therein can be melt processed, they are not generally renewable and not sustainable, which limits the overall sustainability of the film. Unfortunately, polymers that are both biodegradable and renewable are often difficult to melt process into a film.

As such, a need currently exists for a flushable and biodegradable film that has good mechanical properties, and yet is made from low-cost, and abundant raw material, especially when the raw material is a green house gas and is harmful to the climate change.

### SUMMARY OF THE INVENTION

A biodegradable and flushable film is generally provided. In one embodiment, the film can have a thickness of about 50 micrometers or less. The film can be a multi-layer film that includes a water-dispersible core layer that comprises a water-soluble polymer; and a water-barrier skin layer positioned adjacent to the water-dispersible core layer. The water-dispersible core layer can constitute from about 50 wt. % to about 99 wt. % of the film, while the water-barrier skin layer constitutes from about 1 wt. % to about 50 wt. % of the film. The biodegradable polymers of the water-barrier skin layer can constitute from about 80 wt. % to 100 wt. % of the polymer content in the water-barrier skin layer, with from about 10 wt. % to about 90 wt. % of the biodegradable polymers being polyalkylene carbonate and from about 10 wt. % to about 90 wt % of the biodegradable polymers being biodegradable polyesters.

In one embodiment, the biodegradable and flushable film can be included in an absorbent article or a packaging film (e.g., that forms a wrap, pouch, or bag). For example, an absorbent article can include a liquid permeable topsheet; a generally liquid impermeable backsheet; and an absorbent core positioned between the backsheet and the topsheet, with the backsheet including the biodegradable and flushable film.

A method is also generally provided for forming a biodegradable and flushable film. The film can be formed by co-extruding a water-dispersible core layer and a water-barrier skin layer.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a schematic illustration of one embodiment of a method for forming the film of the present invention;

FIG. 2 is a top view of an absorbent article that may be formed in accordance with one embodiment of the present invention;

FIG. 3 shows an SEM Image of Example 1 at 6000×;

FIG. 4 shows an SEM Image of Example 5 at 6000×;

FIG. 5 shows an SEM Image of Example 2 at 6000×;

FIG. 6 shows an SEM Image of Example 6 at 6000×;

FIG. 7 shows an SEM Image of Example 7 at 6000×;

FIG. 8 shows an SEM Image of Example 8 at 6000×; and

FIG. 9 shows an SEM Image of Example 8 at 15000×.

Repeat use of references characters in the present specification and drawings is intended to represent same or analogous features or elements of the invention.

### DETAILED DESCRIPTION OF REPRESENTATIVE EMBODIMENTS

### Definitions

As used herein, the term “biodegradable” generally refers to a material that degrades from the action of naturally occurring microorganisms, such as bacteria, fungi, and algae; environmental heat; moisture; or other environmental factors. The degree of degradation may be determined according to ASTM Test Method 5338.92.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a film that is both biodegradable and flushable, and yet can still act as a barrier to water or other fluids during use. More particularly, the film contains a water-dispersible core layer (i.e., the main layer) that helps the film to lose its integrity after being flushed, as well as a water-barrier skin layer that helps maintain the integrity of the film during use. According to the present invention, the nature and relative concentration of the components in the water-barrier layer are selectively controlled to achieve a combination of different functions. That is, the majority of the polymers employed in the water-barrier layer are biodegradable polymers that can be degraded by microorganisms while in an aqueous environment (e.g., septic tank, wastewater treatment facility, etc.).

According to the present invention, the water-barrier layer of the film contains a combination of one or more polyalkylene carbonate and one or more polyester, where both the polyalkylene carbonate(s) and the polyester(s) are biodegradable and renewable. Despite being biodegradable and renewable, many polyalkylene carbonates tend to be relatively tacky at room temperature, due to their relatively low glass transition temperature in a pure form. For example, polypropylene carbonate are amorphous polymers having a glass transition temperature of about 40° C., while polyethylene carbonate has glass transition temperature of about 25° C. Due to these properties, it was conventionally thought that such polyalkylene carbonates could not be readily formed into films due to its low softening temperature and stickiness and/or tackiness during storage and aging. The present inventors have discovered, however, that through selective control over the components, the film can be readily formed that has good mechanical properties and nonstickiness. Among other things, this is accomplished by blending the polyalkylene carbonate(s) with at least one biodegradable polyester.

Various embodiments of the film layers, as well as the use of the film in certain articles, will now be described in more detail.

### I. Water-Barrier Skin Layer

The water-barrier skin layer of the film is substantially liquid impermeable such that it will effectively limit the flow of liquids therethrough during the time in which it is in use. When the film is employed in an absorbent article, for example, the water-barrier layer may inhibit bodily fluids (e.g., urine) from penetrating through the film and leaking out of the article.

As indicated above, the polymers used to form the water-barrier layer are generally biodegradable in nature. For example, in some embodiments, biodegradable polymers may constitute from about 70 wt. % to 100 wt. %, in some embodiments from about 80 wt. % to 100 wt. %, and in some embodiments, from about 90 wt % to about 99 wt. % of the polymer content of the water-barrier layer. With respect to such polymers, the relative proportion of biodegradable polyesters and polyalkylene carboxylate polymers is also controlled to achieve a balance between biodegradability and melt processability. More specifically, of the biodegradable polymers employed in the water-barrier layer, from about 10 wt. % to about 90 wt. %, in some embodiments from about 15 wt. % to about 55 wt. %, and in some embodiments, from about 20 wt % to about 50 wt. % are typically polyalkylene carbonate polymers. Likewise, the synthetic or bio-based biodegradable polyesters typically constitute from about 10 wt. % to about 90 wt. %, in some embodiments from about 45 wt. % to about 85 wt. %, and in some embodiments, from about 50 wt. % to about 80 wt. % of the biodegradable polymers.

Due to their incompatibility with each other, these components of the water-barrier skin layer can form a layer having different morphologies depending on the method of making the layer and/or the relative amounts of the polyalkylene carbonate and the polyester. As such, unique phase morphological structures can be obtained as desired. For example, in one embodiment, the polyalkylene carbonate polymer can form pockets dispersed within a continuous phase formed by the biodegradable copolyester. This particular morphology can be achieved where the polyalkylene carbonate is at about 60 wt. % or less of the total weight amount of the PAC and the polyester (i.e., where the polyester is about 40 wt. % or more of the total weight amount of the PAC and the polyester). Thus, inversed phase was surprisingly found even where the PAC was a majority by weight of the two components. Alternatively, in another embodiment, the polyalkylene carbonate polymer can form a continuous phase with the biodegradable copolyester forming dispersed domains therein. This particular morphology can be achieved where the polyalkylene carbonate is at about 65 wt. % or more of the total weight amount of the PAC and the polyester (i.e., where the polyester is about 35 wt. % or less of the total weight amount of the PAC and the polyester).

The present inventors have also surprisingly found that the particular method and processing conditions used to form the water-barrier skin layer can affect the morphology of the resulting layer. For example, the morphology of the resulting layer can vary depending on whether the components were melt blended or dry blended to form the layer.

A. Polyalkylene Carbonates

As stated, the water-barrier skin layer of the film can include a polyalkylene carbonate (PAC). Generally, PAC is a copolymer of carbon dioxide and at least one alkylene oxide made by reacting the monomers in presence of a suitable catalyst (e.g. a zinc carboxylate catalyst). Particularly suitable alkylene oxides for use as the at least one alkylene oxide monomer include, but are not limited to, ethylene oxide, propylene oxide, butylene oxide, hexene oxide, octene oxide, cyclopentene oxide, cyclohexene oxide, cis-2-butene oxide, styrene oxide, epichlorohydrin, or mixtures thereof.

As such, the resulting PAC can be a homopolymer or a copolymer of more than one alkylene oxide monomer. Suitable polyalkylene carbonate structures can include repeating alkylene carbonate structure units with 3 to 22 carbonate atoms. Thus, PAC homopolymers can include, but are not limited to, polyethylene carbonate, polypropylene carbonate, polybutylene carbonate, polyhexylene carbonate, etc. PAC copolymers can include two or more different alkyene carbonate structural units (i.e., monomers), such as polyethylene carbonate-co-propylene carbonate, etc. In yet another embodiment, the PAC can be a copolymer of at least one alkylene oxide monomer with other monomer units (e.g., esters, ethers, amide, etc.).

In certain embodiments, the co-polymerization of the alkylene oxide and carbon dioxide can be achieved via heating the alkylene oxide in a solvent at about 40° C. to about 150° C. (e.g., about 60° C. to about 120° C.) for a suitable time in the presence of carbon dioxide and the catalyst(s). The carbon dioxide can be added to the polymerization reaction in a wide range of pressures. However, the pressure of the carbon dioxide is, in one embodiment, at least 100 psig in order to have a useful rate of polymerization. The upper limit of carbon dioxide pressure is limited only by the equipment in which the polymerization is run.

Several catalyst systems are known that catalyze the copolymerization of carbon dioxide and at least one alkylene oxide, such as zinc carboxylate catalysts (e.g., zinc malonate, zinc succinate, zinc glutarate, zinc adipate, zinc hexafluoroglutarate, zinc pimelate, zinc suberate, zinc azelate, zinc sebacate, or mixtures thereof) as described in U.S. Pat. No. 4,789,727, which is incorporated by reference herein. Additional catalysts and systems are disclosed in U.S. Patent Application Publication No. 2011/0309539 of Steinke, et al., U.S. Pat. No. 6,815,529 of Zhao, et al., U.S. Pat. No. 6,599,577 of Zhao, et al., U.S. Publication No. 2002/0082363 of Zhao, et al., U.S. Publication No. 2011/0152497 of Allen, et al., and U.S. Publication No. 2011/0230580 of Allen, et al., all of which are incorporated by reference herein.

The resulting PAC polymer may contain both ether and carbonate linkages in its main chain. The percentage of carbonate linkages can be dependent on a variety of factors, including the reaction conditions and the nature of the catalyst. In one particular embodiment, for example, the PAC polymer can have more than about 85% of carbonate linkages of all linkages between former alkylene oxide monomers.

In certain embodiments, the PAC in the film can have a number average molecular weight (Mₙ) from about 20,000 to about 200,000 g/mol (e.g., from about 30,000 to 100,000 g/mol, such as from about 35,000 to about 80,000 g/mol). Additionally, the PAC can have a weight average molecular weight (“M_{w}”) ranging from about 50,000 to about 300,000 grams per mole, in some embodiments from about 70,000 to about 200,000 grams per mole, and in some embodiments, from about 80,000 to about 150,000 grams per mole. The ratio of the weight average molecular weight to the number average molecular weight (“M_{w}/Mₙ”), i.e., the “polydispersity index”, is also relatively low. For example, the polydispersity index typically ranges from about 1.0 to about 4.0, in some embodiments from about 1.2 to about 3.0, and in some embodiments, from about 1.4 to about 2.0. The weight and number average molecular weights may be determined by methods known to those skilled in the art.

The melt flow index (MI) of the polyalkylene carbonate may generally vary, but is typically in the range of about 0.1 grams per 10 minutes to about 100 grams per 10 minutes, in some embodiments from about 0.5 grams per 10 minutes to about 30 grams per 10 minutes, and in some embodiments, about 1 to about 10 grams per 10 minutes, determined at 190° C. The melt flow index is the weight of the polymer (in grams) that may be forced through an extrusion rheometer orifice (0.0825-inch diameter) when subjected to a force of 2160 grams in 10 minutes at 190° C., and may be determined in accordance with ASTM Test Method D1238-E.

One particularly suitable polyalkylene carbonates for inclusion in the film is polypropylene carbonate (PPC) available from Inner Mongolia Mengxi High-Tech Group Co., Ltd., under the brand name Melicsea with a melt flow of 3.6 g/10 minutes at 150° C.

B. Biodegradable Polyester

The biodegradable polyesters employed in the present invention typically have a relatively low glass transition temperature (“T_{g}”) to reduce stiffness of the film and improve the processability of the polymers. For example, the T₉ may be about 25° C. or less, in some embodiments about 0° C. or less, and in some embodiments, about −10° C. or less. Likewise, the melting point of the biodegradable polyesters is also relatively low to improve the rate of biodegradation. For example, the melting point is typically from about 50° C. to about 180° C., in some embodiments from about 80° C. to about 160° C., and in some embodiments, from about 100° C. to about 140° C. The melting temperature and glass transition temperature may be determined using differential scanning calorimetry (“DSC”) in accordance with ASTM D-3417 as is well known in the art. Such tests may be employed using a DSC Q100 Differential Scanning calorimeter (outfitted with a liquid nitrogen cooling accessory) and with a THERMAL ADVANTAGE (release 4.6.6) analysis software program, which are available from T.A. Instruments Inc. of New Castle, Del.

The biodegradable polyesters may also have a number average molecular weight (“Mₙ”) ranging from about 40,000 to about 120,000 grams per mole, in some embodiments from about 50,000 to about 100,000 grams per mole, and in some embodiments, from about 60,000 to about 85,000 grams per mole. Likewise, the polyesters may also have a weight average molecular weight (“M_{w}”) ranging from about 70,000 to about 300,000 grams per mole, in some embodiments from about 80,000 to about 200,000 grams per mole, and in some embodiments, from about 100,000 to about 150,000 grams per mole. The ratio of the weight average molecular weight to the number average molecular weight (“M_{w}/Mₙ”), i.e., the “polydispersity index”, is also relatively low. For example, the polydispersity index typically ranges from about 1.0 to about 4.0, in some embodiments from about 1.2 to about 3.0, and in some embodiments, from about 1.4 to about 2.0. The weight and number average molecular weights may be determined by methods known to those skilled in the art.

The biodegradable polyesters may also have an apparent viscosity of from about 100 to about 1000 Pascal seconds (Pa·s), in some embodiments from about 200 to about 800 Pa·s, and in some embodiments, from about 300 to about 600 Pa·s, as determined at a temperature of 170° C. and a shear rate of 1000 sec⁻¹. The melt flow index of the biodegradable polyesters may also range from about 0.1 to about 30 grams per 10 minutes, in some embodiments from about 0.5 to about 10 grams per 10 minutes, and in some embodiments, from about 1 to about 5 grams per 10 minutes. The melt flow index is the weight of a polymer (in grams) that may be forced through an extrusion rheometer orifice (0.0825-inch diameter) when subjected to a load of 2160 grams in 10 minutes at a certain temperature (e.g., 190° C.), measured in accordance with ASTM Test Method D1238-E.

Of course, the melt flow index of the biodegradable polyesters will ultimately depend upon the selected film-forming process. For example, when extruded as a cast film, higher melt flow index polymers are typically desired, such as about 2 grams per 10 minutes or more, in some embodiments, from about 4 to about 12 grams per 10 minutes, and in some embodiments, from about 6 to about 9 grams per 10 minutes. Likewise, when formed as a blown film, lower melt flow index polymers are typically desired, such as less than about 12 grams per 10 minutes or less, in some embodiments from about 1 to about 7 grams per 10 minutes, and in some embodiments, from about 2 to about 5 grams per 10 minutes.

Examples of suitable biodegradable polyesters include aliphatic polyesters, such as polycaprolactone, polyesteramides, modified polyethylene terephthalate, polylactic acid (PLA) and its copolymers, terpolymers based on polylactic acid, polyglycolic acid, polyalkylene carbonates (such as polyethylene carbonate), polyhydroxyalkanoates (PHA), poly-3-hydroxybutyrate (PHB), poly-3-hydroxyvalerate (PHV), poly-3-hydroxybutyrate-co-4-hydroxybutyrate, poly-3-hydroxybutyrate-co-3-hydroxyvalerate copolymers (PHBV), poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, and succinate-based aliphatic polymers (e.g., polybutylene succinate, polybutylene succinate adipate, polyethylene succinate, etc.); aromatic polyesters and modified aromatic polyesters; and aliphatic-aromatic copolyesters. In one particular embodiment, the biodegradable polyester is an aliphatic-aromatic copolyester (e.g., block, random, graft, etc.). The aliphatic-aromatic copolyester may be synthesized using any known technique, such as through the condensation polymerization of a polyol in conjunction with aliphatic and aromatic dicarboxylic acids or anhydrides thereof. The polyols may be substituted or unsubstituted, linear or branched, polyols selected from polyols containing 2 to about 12 carbon atoms and polyalkylene ether glycols containing 2 to 8 carbon atoms. Examples of polyols that may be used include, but are not limited to, ethylene glycol, diethylene glycol, propylene glycol, 1,2-propanediol, 1,3-propanediol, 2,2-dimethyl-1,3-propanediol, 1,2-butanediol, 1,3-butanediol, 1,4-butanediol, 1,2-pentanediol, 1,5-pentanedial, 1,6-hexanediol, polyethylene glycol, diethylene glycol, 2,2,4-trimethyl-1,6-hexanediol, thiodiethanol, 1,3-cyclohexanedimethanol, 1,4-cyclohexanedimethanol, 2,2,4,4-tetramethyl-1,3-cyclobutanediol, cyclopentanediol, triethylene glycol, and tetraethylene glycol. Preferred polyols include 1,4-butanediol; 1,3-propanediol; ethylene glycol; 1,6-hexanediol; diethylene glycol; and 1,4-cyclohexanedimethanol.

Representative aliphatic dicarboxylic acids that may be used include substituted or unsubstituted, linear or branched, non-aromatic dicarboxylic acids selected from aliphatic dicarboxylic acids containing 1 to about 10 carbon atoms, and derivatives thereof. Non-limiting examples of aliphatic dicarboxylic acids include malonic, malic, succinic, oxalic, glutaric, adipic, pimelic, azelaic, sebacic, fumaric, 2,2-dimethyl glutaric, suberic, 1,3-cyclopentanedicarboxylic, 1,4-cyclohexanedicarboxylic, 1,3-cyclohexanedicarboxylic, diglycolic, itaconic, maleic, and 2,5-norbornanedicarboxylic. Representative aromatic dicarboxylic acids that may be used include substituted and unsubstituted, linear or branched, aromatic dicarboxylic acids selected from aromatic dicarboxylic acids containing 8 or more carbon atoms, and derivatives thereof. Non-limiting examples of aromatic dicarboxylic acids include terephthalic acid, dimethyl terephthalate, isophthalic acid, dimethyl isophthalate, 2,6-napthalene dicarboxylic acid, dimethyl-2,6-naphthalate, 2,7-naphthalenedicarboxylic acid, dimethyl-2,7-naphthalate, 3,4′-diphenyl ether dicarboxylic acid, dimethyl-3,4′ diphenyl ether dicarboxylate, 4,4′-diphenyl ether dicarboxylic acid, dimethyl-4,4′-diphenyl ether dicarboxylate, 3,4′-diphenyl sulfide dicarboxylic acid, dimethyl-3,4′-diphenyl sulfide dicarboxylate, 4,4′-diphenyl sulfide dicarboxylic acid, dimethyl-4,4′-diphenyl sulfide dicarboxylate, 3,4′-diphenyl sulfone dicarboxylic acid, dimethyl-3,4′-diphenyl sulfone dicarboxylate, 4,4′-diphenyl sulfone dicarboxylic acid, dimethyl-4,4′-diphenyl sulfone dicarboxylate, 3,4′-benzophenonedicarboxylic acid, dimethyl-3,4′-benzophenonedicarboxylate, 4,4′-benzophenonedicarboxylic acid, dimethyl-4,4′-benzophenonedicarboxylate, 1,4-naphthalene dicarboxylic acid, dimethyl-1,4-naphthalate, 4,4′-methylene bis(benzoic acid), dimethyl-4,4′-methylenebis(benzoate), etc., and mixtures thereof.

The polymerization may be performed in the presence of a catalyst, such as a titanium-based catalyst (e.g., tetraisopropyltitanate, tetraisopropoxy titanium, dibutoxydiacetoacetoxy titanium, or tetrabutyltitanate). If desired, a diisocyanate chain extender may be reacted with the copolyester to increase its molecular weight. Representative diisocyanates may include toluene 2,4-diisocyanate, toluene 2,6-diisocyanate, 2,4′-diphenylmethane diisocyanate, naphthylene-1,5-diisocyanate, xylylene diisocyanate, hexamethylene diisocyanate (“HMDI”), isophorone diisocyanate and methylenebis(2-isocyanatocyclohexane). Trifunctional isocyanate compounds may also be employed that contain isocyanurate and/or biurea groups with a functionality of not less than three, or to replace the diisocyanate compounds partially by tri-or polyisocyanates. The preferred diisocyanate is hexamethylene diisocyanate. The amount of the chain extender employed is typically from about 0.3 to about 3.5 wt. %, in some embodiments, from about 0.5 to about 2.5 wt. % based on the total weight percent of the polymer.

The copolyesters may either be a linear polymer or a long-chain branched polymer. Long-chain branched polymers are generally prepared by using a low molecular weight branching agent, such as a polyol, polycarboxylic acid, hydroxy acid, and so forth. Representative low molecular weight polyols that may be employed as branching agents include glycerol, trimethylolpropane, trimethylolethane, polyethertriols, 1,2,4-butanetriol, pentaerythritol, 1,2,6-hexanetriol, sorbitol, 1,1,4,4,-tetrakis(hydroxymethyl)cyclohexane, tris(2-hydroxyethyl) isocyanurate, and dipentaerythritol. Representative higher molecular weight polyols (molecular weight of 400 to 3000) that may be used as branching agents include triols derived by condensing alkylene oxides having 2 to 3 carbons, such as ethylene oxide and propylene oxide with polyol initiators. Representative polycarboxylic acids that may be used as branching agents include hemimellitic acid, trimellitic (1,2,4-benzenetricarboxylic) acid and anhydride, trimesic (1,3,5-benzenetricarboxylic) acid, pyromellitic acid and anhydride, benzenetetracarboxylic acid, benzophenone tetracarboxylic acid, 1,1,2,2-ethane-tetracarboxylic acid, 1,1,2-ethanetricarboxylic acid, 1,3,5-pentanetricarboxylic acid, and 1,2,3,4-cyclopentanetetracarboxylic acid. Representative hydroxy acids that may be used as branching agents include malic acid, citric acid, tartaric acid, 3-hydroxyglutaric acid, mucic acid, trihydroxyglutaric acid, 4-carboxyphthalic anhydride, hydroxylsophthalic acid, and 4-(beta-hydroxyethyl)phthalic acid. Such hydroxy acids contain a combination of 3 or more hydroxyl and carboxyl groups. Especially preferred branching agents include trimellitic acid, trimesic acid, pentaerythritol, trimethylol propane and 1,2,4-butanetriol.

The aromatic dicarboxylic acid monomer constituent may be present in the copolyester in an amount of from about 10 mole % to about 40 mole %, in some embodiments from about 15 mole % to about 35 mole %, and in some embodiments, from about 15 mole % to about 30 mole %. The aliphatic dicarboxylic acid monomer constituent may likewise be present in the copolyester in an amount of from about 15 mole % to about 45 mole %, in some embodiments from about 20 mole % to about 40 mole %, and in some embodiments, from about 25 mole % to about 35 mole %. The polyol monomer constituent may also be present in the aliphatic-aromatic copolyester in an amount of from about 30 mole % to about 65 mole %, in some embodiments from about 40 mole % to about 50 mole %, and in some embodiments, from about 45 mole % to about 55 mole %.

In one particular embodiment, for example, the aliphatic-aromatic copolyester may comprise the following structure:

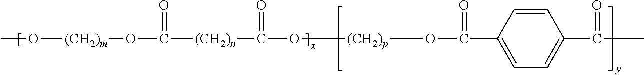

wherein,

m is an integer from 2 to 10, in some embodiments from 2 to 4, and in one embodiment, 4;

n is an integer from 0 to 18, in some embodiments from 2 to 4, and in one embodiment, 4;

p is an integer from 2 to 10, in some embodiments from 2 to 4, and in one embodiment, 4;

x is an integer greater than 1; and

y is an integer greater than 1. One example of such a copolyester is polybutylene adipate terephthalate, which is commercially available under the designation ECOFLEX® F BX 7011 from BASF Corp. Another example of a suitable copolyester containing an aromatic terephtalic acid monomer constituent is available under the designation ENPOL™ 8060M from IRE Chemicals (South Korea). Other suitable aliphatic-aromatic copolyesters may be described in U.S. Pat. Nos. 5,292,783; 5,446,079; 5,559,171; 5,580,911; 5,599,858; 5,817,721; 5,900,322; and 6,258,924, which are incorporated herein in their entirety by reference thereto for all purposes. Still other suitable biodegradable polyesters are described in U.S. Pat. No. 6,472,497 to Loercks, et al. and U.S. Patent Application Publication No. 2005/0182196 to Khemani, et al., which are incorporated herein in their entirety by reference thereto for all relevant purposes.

C. Other Components

One beneficial aspect of the present invention is that a film can be readily formed without the need for compatibilizers or plasticizers conventionally thought to be required to melt process a polyalkylene carbonate, Thus, in certain embodiments, the film layer may be free of such ingredients, which further enhances the overall biodegradability and renewability of the film. Additionally, in one embodiment, the film can be free from other polymeric material.

Nevertheless, in some cases, compatibilizer and/or plasticizers may still be employed in the film layer, typically in an amount of no more than about 40 wt. %, in some embodiments from about 0.1 wt. % to about 30 wt. %, in some embodiments from about 0.5 wt. % to about 25 wt. %, and in some embodiments, from about 1 wt. % to about 15 wt. % of the film layer.

When employed, the compatibilizer can include a component compatible with the polyalkylene carbonate and another component compatible with the polyester. For example, the compatibilizer may be a functionalized polyester or polyolefin that possesses a polar component provided by one or more functional groups that is compatible with the polyalkylene carbonates and a non-polar component provided by an olefin that is compatible with the polyester. The polar component may, for example, be provided by one or more functional groups and the non-polar component may be provided by an olefin. The olefin component of the compatibilizer may generally be formed from any linear or branched α-olefin monomer, oligomer, or polymer (including copolymers) derived from an olefin monomer. The α-olefin monomer typically has from 2 to 14 carbon atoms and preferably from 2 to 6 carbon atoms. Examples of suitable monomers include, but not limited to, ethylene, propylene, butene, pentene, hexene, 2-methyl-1-propene, 3-methyl-1-pentene, 4-methyl-1-pentene, and 5-methyl-1-hexene. Examples of polyolefins include both homopolymers and copolymers, i.e., polyethylene, ethylene copolymers such as EPDM, polypropylene, propylene copolymers, and polymethylpentene polymers. An olefin copolymer can include a minor amount of non-olefinic monomers, such as styrene, vinyl acetate, diene, or acrylic and non-acrylic monomer. Functional groups may be incorporated into the polymer backbone using a variety of known techniques. For example, a monomer containing the functional group may be grafted onto a polyolefin backbone to form a graft copolymer. Such grafting techniques are well known in the art and described, for instance, in U.S. Pat. No. 5,179,164. In other embodiments, the monomer containing the functional groups may be copolymerized with an olefin monomer to form a block or random copolymer. Regardless of the manner in which it is incorporated, the functional group of the compatibilizer may be any group that provides a polar segment to the molecule, such as a carboxyl group, acid anhydride group, acid amide group, imide group, carboxylate group, epoxy group, amino group, isocyanate group, group having oxazoline ring, hydroxyl group, and so forth. Maleic anhydride modified polyolefins are particularly suitable for use in the present invention. Such modified polyolefins are typically formed by grafting maleic anhydride onto a polymeric backbone material. Such maleated polyolefins are available from E. I. du Pont de Nemours and Company under the designation Fusabond®, such as the P Series (chemically modified polypropylene), E Series (chemically modified polyethylene), C Series (chemically modified ethylene vinyl acetate), A Series (chemically modified ethylene acrylate copolymers or terpolymers), or N Series (chemically modified ethylene-propylene, ethylene-propylene diene monomer (“EPDM”) or ethylene-octene). Alternatively, maleated polyolefins are also available from Chemtura Corp. under the designation Polybond® and Eastman Chemical Company under the designation Eastman G series, and AMPLIFYT™ GR Functional Polymers (maleic anhydride grafted polyolefins). As stated, grafted polyesters may also be included, and may include for example the grafted biodegradable aliphatic polyesters and aliphatic aromatic copolyesters as described above.

Likewise, when employed, suitable plasticizers may include polyhydric alcohol plasticizers, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, and sorbitol), polyols (e.g., ethylene glycol, glycerol, propylene glycol, dipropylene glycol, butylene glycol, and hexane triol), etc. Also suitable are hydrogen bond-forming organic compounds which do not have hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable plasticizers may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids may also be used, such as copolymers of ethylene and acrylic acid, polyethylene grafted with maleic acid, polybutadiene-co-acrylic acid, polybutadiene-co-maleic acid, polypropylene-co-acrylic acid, polypropylene-co-maleic acid, and other hydrocarbon based acids. A low molecular weight plasticizer is preferred, such as less than about 20,000 g/mol, preferably less than about 5,000 g/mol and more preferably less than about 1,000 g/mol.

Besides the components noted above, still other additives may also be incorporated into the film, such as melt stabilizers, dispersion aids (e.g., surfactants), processing aids (PPA) or stabilizers, heat stabilizers, light stabilizers, antioxidants, heat aging stabilizers, whitening agents, antiblocking agents, bonding agents, lubricants, fillers, anti-static additives, etc.

For example, a dispersion aids can improve the water dispersibility of the layer. When employed, the dispersion aid(s) typically constitute from about 0.01 wt. % to about 10 wt. %, in some embodiments from about 0.1 wt. % to about 5 wt. %, and in some embodiments, from about 0.5 wt. % to about 4 wt. % of the polyalkylene carbonate.

Although any dispersion aid may generally be employed in the present invention, surfactants having a certain hydrophilic/lipophilic balance (“HLB”) may improve the long-term stability of the composition. The HLB index is well known in the art and is a scale that measures the balance between the hydrophilic and lipophilic solution tendencies of a compound. The HLB scale ranges from 1 to approximately 50, with the lower numbers representing highly lipophilic tendencies and the higher numbers representing highly hydrophilic tendencies. In some embodiments of the present invention, the HLB value of the surfactants is from about 1 to about 20, in some embodiments from about 1 to about 15 and in some embodiments, from about 2 to about 10. If desired, two or more surfactants may be employed that have HLB values either below or above the desired value, but together have an average HLB value within the desired range.

One particularly suitable class of surfactants for use in the present invention are nonionic surfactants, which typically have a hydrophobic base (e.g., long chain alkyl group or an alkylated aryl group) and a hydrophilic chain (e.g., chain containing ethoxy and/or propoxy moieties). For instance, some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain carboxylic acids, and mixtures thereof. In one particular embodiment, the nonionic surfactant may be a fatty acid ester, such as a sucrose fatty acid ester, glycerol fatty acid ester, propylene glycol fatty acid ester, sorbitan fatty acid ester, pentaerythritol fatty acid ester, sorbitol fatty acid ester, and so forth. The fatty acid used to form such esters may be saturated or unsaturated, substituted or unsubstituted, and may contain from 6 to 22 carbon atoms, in some embodiments from 8 to 18 carbon atoms, and in some embodiments, from 12 to 14 carbon atoms. In one particular embodiment, mono- and di-glycerides of fatty acids may be employed in the present invention.

### II. Water-Dispersible Core Layer

The water-dispersible layer of the film will generally break apart into smaller pieces or completely dissolve when placed in an aqueous environment. The amount of time needed for dispersal of the water-dispersible layer will typically depend at least in part upon the particular end-use design criteria. Typically, the water-dispersible layer will be fully dispersed within the aqueous environment within about 60 minutes, suitably within about 15 minutes, more suitably within about 5 minutes, and most suitably within about 30 seconds.

A. Water-Soluble Polymer

To impart the desired degree of dispersibility, the water-dispersible layer includes at least one water-soluble polymer. The water-soluble polymer may be formed from monomers such as vinyl pyrrolidone, hydroxyethyl acrylate or methacrylate (e.g., 2-hydroxyethyl methacrylate), hydroxypropyl acrylate or methacrylate, acrylic or methacrylic acid, acrylic or methacrylic esters or vinyl pyridine, acrylamide, vinyl acetate, vinyl alcohol, ethylene oxide, derivatives thereof, and so forth. Other examples of suitable monomers are described in U.S. Pat. No. 4,499,154 to James, et al., which is incorporated herein in its entirety by reference thereto for all purposes. The resulting polymers may be homopolymers or interpolymers (e.g., copolymer, terpolymer, etc.), and may be nonionic, anionic, cationic, or amphoteric. In addition, the polymer may be of one type (i.e., homogeneous), or mixtures of different polymers may be used (i.e., heterogeneous). In one particular embodiment, the water-soluble polymer contains a repeating unit having a functional hydroxyl group, such as polyvinyl alcohol (“PVOH”), copolymers of polyvinyl alcohol (e.g., ethylene vinyl alcohol copolymers, methyl methacrylate vinyl alcohol copolymers, etc.), etc.

Vinyl alcohol polymers, for instance, have at least two or more vinyl alcohol units in the molecule and may be a homopolymer of vinyl alcohol, or a copolymer containing other monomer units. Vinyl alcohol homopolymers may be obtained by complete hydrolysis of a vinyl alkanoate polymer, such as vinyl formate, vinyl acetate, vinyl propionate, etc. Vinyl alcohol copolymers may be obtained by incomplete hydrolysis of a vinyl alkanoate with an olefin having 2 to 30 carbon atoms, such as ethylene, propylene, 1-butene, etc.; an unsaturated carboxylic acid having 3 to 30 carbon atoms, such as acrylic acid, methacrylic acid, crotonic acid, maleic acid, fumaric acid, etc., or an ester, salt, anhydride or amide thereof; an unsaturated nitrile having 3 to 30 carbon atoms, such as acrylonitrile, methacrylonitrile, etc.; a vinyl ether having 3 to 30 carbon atoms, such as methyl vinyl ether, ethyl vinyl ether, etc.; and so forth. The degree of hydrolysis may be selected to optimize solubility, etc., of the polymer. For example, the degree of hydrolysis may be from about 60 mole % to about 95 mole %, in some embodiments from about 80 mole % to about 90 mole %, and in some embodiments, from about 85 mole % to about 89 mole %. Examples of suitable partially hydrolyzed polyvinyl alcohol polymers are available under the designation CELVOL™ 203, 205, 502, 504, 508, 513, 518, 523, 530, or 540 from Celanese Corp. Other suitable partially hydrolyzed polyvinyl alcohol polymers are available under the designation ELVANOL™ 50-14, 50-26, 50-42, 51-03, 51-04, 51-05, 51-08, and 52-22 from DuPont.

In one embodiment, the water-soluble core layer comprises of a vinyl alcohol polymer, a plasticizer, and an inorganic filler (such as described below in subsection II.D).

B. Biodegradable Polymers

If desired, biodegradable polymers may also be employed in the water-dispersible layer to enhance its biodegradability under storage conditions. The biodegradable polymers may or may not be water soluble. For instance, suitable water-soluble biodegradable polymers may include a native starch and chemically modified starch polymers (e.g., starch ethers, such as hydroxyalkyl starch, starch esters such as starch acetate, etc.). Starch is a natural polymer composed of amylose and amylopectin. Amylose is essentially a linear polymer having a molecular weight in the range of 100,000-500,000, whereas amylopectin is a highly branched polymer having a molecular weight of up to several million. Although starch is produced in many plants, typical sources includes seeds of cereal grains, such as corn, waxy corn, wheat, sorghum, rice, and waxy rice; tubers, such as potatoes; roots, such as tapioca (i.e., cassava and manioc), sweet potato, and arrowroot; and the pith of the sago palm.

Broadly speaking, any natural (unmodified) and/or modified starch having the desired water sensitivity properties may be employed in the present invention. Modified starches, for instance, are often employed that have been chemically modified by typical processes known in the art (e.g., esterification, etherification, oxidation, acid hydrolysis, enzymatic hydrolysis, etc.). Starch ethers and/or esters may be particularly desirable, such as hydroxyalkyl starches, carboxymethyl starches, etc. The hydroxyalkyl group of hydroxylalkyl starches may contain, for instance, 1 to 10 carbon atoms, in some embodiments from 1 to 6 carbon atoms, in some embodiments from 1 to 4 carbon atoms, and in some embodiments, from 2 to 4 carbon atoms. Representative hydroxyalkyl starches such as hydroxyethyl starch, hydroxypropyl starch, hydroxybutyl starch, and derivatives thereof. Starch esters, for instance, may be prepared using a wide variety of anhydrides (e.g., acetic, propionic, butyric, and so forth), organic acids, acid chlorides, or other esterification reagents. The degree of esterification may vary as desired, such as from 1 to 3 ester groups per glucosidic unit of the starch.

A plasticizer is also typically employed in the thermoplastic starch to render the starch melt-processible. Starches normally exist in the form of granules that have a coating or outer membrane that encapsulates the more water-soluble amylose and amylopectin chains within the interior of the granule. When heated, polar solvents (“plasticizers”) may soften and penetrate the outer membrane and cause the inner starch chains to absorb water and swell. This swelling will, at some point, cause the outer shell to rupture and result in an irreversible destructurization of the starch granule. Once destructurized, the starch polymer chains containing amylose and amylopectin polymers, which are initially compressed within the granules, will stretch out and form a generally disordered intermingling of polymer chains. Upon resolidification, however, the chains may reorient themselves to form crystalline or amorphous solids having varying strengths depending on the orientation of the starch polymer chains. Because the starch (natural or modified) is thus capable of melting and resolidifying, it is generally considered a “thermoplastic starch.”

Suitable plasticizers may include, for instance, polyhydric alcohol plasticizers, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, glycerol, and sorbitol), polyols (e.g., ethylene glycol, propylene glycol, dipropylene glycol, butylene glycol, and hexane triol), etc. Also suitable are hydrogen bond forming organic compounds which do not have hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable plasticizers may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids may also be used, such as ethylene acrylic acid, ethylene maleic acid, butadiene acrylic acid, butadiene maleic acid, propylene acrylic acid, propylene maleic acid, and other hydrocarbon based acids. A low molecular weight plasticizer is preferred, such as less than about 20,000 g/mol, preferably less than about 5,000 g/mol and more preferably less than about 1,000 g/mol.

The relative amount of starches and plasticizers employed in the thermoplastic starch may vary depending on a variety of factors, such as the molecular weight of the starch, the type of starch (e.g., modified or unmodified), the affinity of the plasticizer for the starch, etc. Typically, however, starches constitute from about 40 wt. % to about 95 wt. %, in some embodiments from about 50 wt. % to about 90 wt. %, and in some embodiments, from about 60 wt. % to about 80 wt. % of the thermoplastic composition. Likewise, plasticizers typically constitute from about 5 wt. % to about 60 wt. %, in some embodiments from about 10 wt. % to about 50 wt. %, and in some embodiments, from about 20 wt. % to about 40 wt. % of the thermoplastic composition. It should be understood that the weight of starch referenced herein includes any bound water that naturally occurs in the starch before mixing it with other components to form the thermoplastic starch. Starches, for instance, typically have a bound water content of about 5% to 16% by weight of the starch.

Likewise, biodegradable polymers that are not water soluble may include the aforementioned biodegradable polyesters (e.g., aliphatic polyesters, aliphatic-aromatic copolyesters, etc.). Combinations of such polymers may also be employed. When employed, the biodegradable polymers typically constitute from about 50 wt. % to about 95 wt. %, in some embodiments from about 60 wt. % to about 90 wt. %, and in some embodiments, from about 70 wt. % to about 85 wt. % of the polymer content of the water-dispersible layer. Likewise, in such embodiments, water-soluble polymers typically constitute from about 5 wt. % to about 50 wt. %, in some embodiments from about 10 wt. % to about 40 wt. %, and in some embodiments, from about 15 wt. % to about 30 wt. % of the polymer content of the layer.

In one particular embodiment, for example, the water-dispersible layer contains a combination of water-soluble polymers (e.g., polyvinyl alcohol), starch polymers (e.g., chemically modified starch), and biodegradable polyesters (e.g., aliphatic polyesters, aliphatic-aromatic copolyesters, etc.). In such embodiments, the starch polymers may constitute from about 30 wt. % to about 70 wt. %, in some embodiments from about 40 wt. % to about 60 wt. %, and in some embodiments, from about 45 wt. % to about 55 wt. % of the polymer content of the layer, and the biodegradable polyesters may constitute from about 10 wt. % to about 40 wt. %, in some embodiments from about 15 wt. % to about 35 wt. %, and in some embodiments, from about 20 wt. % to about 30 wt. % of the polymer content of the layer.

In another embodiment, for example, the water-dispersible layer contains a combination of water-soluble polymers (e.g., polyvinyl alcohol) and biodegradable polyesters (e.g., aliphatic polyesters, aliphatic-aromatic copolyesters, etc.). In such embodiments, the water-soluble polymers may constitute from about 50 wt. % to about 95 wt. %, in some embodiments from about 60 wt. % to about 90 wt. %, and in some embodiments, from about 70 wt. % to about 85 wt. % of the polymer content of the layer, and the biodegradable polyesters may constitute from about 10 wt. % to about 50 wt. %, in some embodiments from about 10 wt. % to about 40 wt. %, and in some embodiments, from about 15 wt. % to about 30 wt. % of the polymer content of the layer.

C. Plasticizers

Plasticizers may also be employed in certain embodiments of the water-dispersible layer. Suitable plasticizers may include those described above, such as polyhydric alcohols. When employed, plasticizers typically constitute from about 5 wt. % to about 50 wt. %, in some embodiments from about 10 wt. % to about 40 wt. %, and in some embodiments, from about 15 wt. % to about 30 wt. % of the water-dispersible layer. In one particular embodiment, for example, the water-dispersible layer contains a combination of water-soluble polymers (e.g., polyvinyl alcohol) and a plasticizer. The water-soluble polymers may constitute from about 50 wt. % to about 95 wt. %, in some embodiments from about 60 wt. % to about 90 wt. %, and in some embodiments, from about 70 wt. % to about 85 wt. % of the water-dispersible layer, and the plasticizer may constitute from about 5 wt. % to about 50 wt. %, in some embodiments from about 10 wt. % to about 40 wt. %, and in some embodiments, from about 15 wt. % to about 30 wt. % of the polymer content of the layer.

D. Fillers

If desired, fillers may also be employed in the water-dispersible layer. Fillers are particulates or other forms of material that may be added to the film polymer extrusion blend and that will not chemically interfere with the extruded film, but which may be uniformly dispersed throughout the film. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways. Breathable microporous elastic films are described, for example, in U.S. Pat. Nos. 5,997,981; 6,015,764; and 6,111,163 to McCormack, et al.; 5,932,497 to Morman, et al.; 6,461,457 to Taylor, et al., which are incorporated herein in their entirety by reference thereto for all purposes. Further, hindered phenols are commonly used as an antioxidant in the production of films. Some suitable hindered phenols include those available from Ciba Specialty Chemicals under the trade name “Irganox®”, such as Irganox® 1076, 1010, or E 201. Moreover, bonding agents may also be added to the film to facilitate bonding of the film to additional materials (e.g., nonwoven webs). Examples of such bonding agents include hydrogenated hydrocarbon resins. Other suitable bonding agents are described in U.S. Pat. Nos. 4,789,699 to Kieffer et al. and 5,695,868 to McCormack, which are incorporated herein in their entirety by reference thereto for all purposes.

When employed, the filler may include particles having any desired size, such as those having an average size of from about 0.5 to about 10 micrometers, in some embodiments, from about 1 to about 8 micrometers, and in some embodiments, from about 2 to about 6 micrometers. Suitable particles for use as a filler may include inorganic oxides, such as calcium carbonate, kaolin clay, silica, alumina, barium carbonate, sodium carbonate, titanium dioxide, zeolites, magnesium carbonate, calcium oxide, magnesium oxide, aluminum hydroxide, talc, etc.; sulfates, such as barium sulfate, magnesium sulfate, aluminum sulfate, etc.; cellulose-type powders (e.g., pulp powder, wood powder, etc.); carbon; cyclodextrins; synthetic polymers (e.g., polystyrene), and so forth. Still other suitable particles are described in U.S. Pat. Nos. 6,015,764 and 6,111,163 to McCormack, et al.; 5,932,497 to Morman, et al.; 5,695,868 to McCormack; 5,855,999 to McCormack, et al.; 5,997,981 to McCormack et al.; and 6,461,457 to Taylor, et al., which are incorporated herein in their entirety by reference thereto for all purposes.

Although not required, the filler may optionally be coated with a modifier (e.g., fatty acid, such as stearic acid or behenic acid) to facilitate the free flow of the particles in bulk and their ease of dispersion into the composition. Further, the filler may also be coated with a liquid additive to reduce coupling at the resin-filler interface and facilitate debonding of filler from polymer matrix during stretching. This is especially useful for the polar biodegradable polymers, which demonstrate strong interaction with fillers. Examples of such additives include surfactants, such as silicone glycol copolymers available from Dow Corning Corporation. Other suitable additives for this purpose may include titanates available from Kenrich Petrochemicals, Inc. of Bayonne, N.J. under the designations Ken-React® LICA® 01, React® LICA® 12, Ken-React® CAPOW®, Ken-React® CAPS® and zirconates available from Kenrich under the designation Ken-React® CAPS NZ 01/L. The filler may be pre-compounded with such additives before mixing with the resin, or the additives may be compounded with the resin and fillers at the melt-blending step.

In one particular embodiment, for example, the water-dispersible layer contains a combination of water-soluble polymers and a filler. In such embodiments, the filler typically constitutes from about 1 wt. % to about 30 wt. %, in some embodiments from about 2 wt. % to about 25 wt. %, and in some embodiments, from about 5 wt. % to about 20 wt. % of the water-dispersible layer. Likewise, water-soluble polymers typically constitute from about 70 wt. % to about 99 wt. %, in some embodiments from about 75 wt. % to about 98 wt. %, and in some embodiments, from about 80 wt. % to about 95 wt. % of the water-dispersible layer.

In another embodiment, the water-dispersible layer comprises a combination of water-soluble polymer(s) (e.g., a polyvinyl alcohol polymer), a biodegradable polymer(s) (e.g., a biodegradable polyester), a modified starch polymer (e.g., a hydroxyalkyl starch polymer), a plasticizer, and a filler.

E. Additional Additives

Besides the components noted above, still other additives may also be incorporated into the water-dispersible layer, such as melt stabilizers, processing stabilizers, heat stabilizers, light stabilizers, antioxidants, heat aging stabilizers, whitening agents, antiblocking agents, bonding agents, lubricants etc.

### II. Film Construction

The film of the present invention contains a water-dispersible core layer that is positioned adjacent to a water-barrier skin layer. Of course, a variety of other layers may also be employed in the film as desired. In one embodiment, for example, it may be desirable to employ two skin layers that sandwich the core layer. If desired, both of the skin layers may be formed as a biodegradable water-barrier in the manner described herein. Alternatively, one skin layer may be formed from different components, such as traditional film-forming materials (e.g., polyolefin). Regardless of the number of layers, however, the water-dispersible core layer typically constitutes a substantial portion of the weight of the film, such as from about 50 wt. % to about 99 wt. %, in some embodiments from about 55 wt. % to about 90 wt. %, and in some embodiments, from about 60 wt. % to about 85 wt. % of the film. On the other hand, the skin layer(s) such as from about 1 wt. % to about 50 wt. %, in some embodiments from about 5 wt. % to about 30 wt. %, and in some embodiments, from about 10 wt. % to about 20 wt. % of the film.

Each skin layer may also have a thickness of from about 0.1 to about 10 micrometers, in some embodiments from about 0.5 to about 5 micrometers, and in some embodiments, from about 1 to about 2.5 micrometers. Likewise, the core layer may have a thickness of from about from about 1 to about 40 micrometers, in some embodiments from about 2 to about 25 micrometers, and in some embodiments, from about 5 to about 20 micrometers. The total thickness of the film may generally vary depending upon the desired use. Nevertheless, the film thickness is typically minimized to reduce the time needed for the film to disperse in water. Thus, in most embodiments of the present invention, the film has a total thickness of about 50 micrometers or less, in some embodiments from about 1 to about 40 micrometers, in some embodiments from about 2 to about 35 micrometers, and in some embodiments, from about 5 to about 30 micrometers.

Despite having such a small thickness and good sensitivity in water, the film of the present invention is nevertheless able to retain good dry mechanical properties during use. One parameter that is indicative of the relative dry strength of the film is the ultimate tensile strength, which is equal to the peak stress obtained in a stress-strain curve. Desirably, the film of the present invention exhibits an ultimate tensile strength in the machine direction (“MD”) of from about 10 to about 80 Megapascals (MPa), in some embodiments from about 15 to about 60 MPa, and in some embodiments, from about 20 to about 50 MPa, and an ultimate tensile strength in the cross-machine direction (“CD”) of from about 2 to about 40 Megapascals (MPa), in some embodiments from about 4 to about 40 MPa, and in some embodiments, from about 5 to about 30 MPa. Although possessing good strength, it is also desirable that the film is not too stiff. One parameter that is indicative of the relative stiffness of the film (when dry) is Young's modulus of elasticity, which is equal to the ratio of the tensile stress to the tensile strain and is determined from the slope of a stress-strain curve. For example, the film typically exhibits a Young's modulus in the machine direction (“MD”) of from about 50 to about 1200 Megapascals (“MPa”), in some embodiments from about 100 to about 800 MPa, and in some embodiments, from about 150 to about 600 MPa, and a Young's modulus in the cross-machine direction (“CD”) of from about 50 to about 1000 Megapascals (“MPa”), in some embodiments from about 100 to about 800 MPa, and in some embodiments, from about 150 to about 500 MPa. The MD elongation of the film may also be about 40% or more, in some embodiments about 60% or more, and in some embodiments, about 80% or more.

Other properties of the resulting film may generally vary as desired. For example, depending on the intended application, the film may be generally liquid and vapor-impermeable or generally liquid impermeable, yet vapor-permeable (i.e., “breathable”). Breathable films, for example, are often used in absorbent articles (e.g., outer cover) in which it is desired to allow moisture to escape from the absorbent core through the film. Breathable films may be formed with the use of a filler, such as described above. Filled films may be made breathable by stretching, which causes the polymer to break away from the filler and create microporous passageways. Techniques for forming microporous films are described, for example, in U.S. Pat. No. 7,153,569 to Kaufman, et al., as well as U.S. Application Publication Nos. 2005/0208294 to Kaufman, et al. and 2006/0149199 to Topolkaraev, et al., all of which are incorporated herein in their entirety by reference thereto for all purposes.

In embodiments in which it is desired to impart breathability, the film typically exhibits a water vapor transmission rate (WVTR) of about 800 grams/m²-24 hours or more, in some embodiments about 1,000 grams/m²-24 hours or more, in some embodiments about 1,200 grams/m²-24 hours or more, and in some embodiments, from about 1,500 to about 10,000 grams/m²-24 hours. The film may also limit the amount of liquid water that passes therethrough upon the application of pressure, i.e., it resists a hydrostatic pressure (“hydrohead”) of about 50 millibar or more, in some embodiments about 70 millibar or more, in some embodiments about 80 millibar or more, and in some embodiments, about 100 millibar or more without allowing liquid water to pass.

The multi-layered film of the present invention may be prepared by co-extrusion of the layers, extrusion coating, or by any conventional layering process. Two particularly advantageous processes are cast film coextrusion processes and blown film coextrusion processes. In such processes, two or more of the film layers are formed simultaneously and exit the extruder in a multilayer form. Some examples of such processes are described in U.S. Pat. Nos. 6,075,179 to McCormack, et al. and 6,309,736 to McCormack, et al., which are incorporated herein in their entirety by reference thereto for all purposes. Processes for producing blown films are likewise described, for instance, in U.S. Pat. Nos. 3,354,506 to Raley; 3,650,649 to Schippers; and 3,801,429 to Schrenk et al., as well as U.S. Patent Application Publication Nos. 2005/0245162 to McCormack, et al. and 2003/0068951 to Bows, et al., all of which are incorporated herein in their entirety by reference thereto for all purposes.

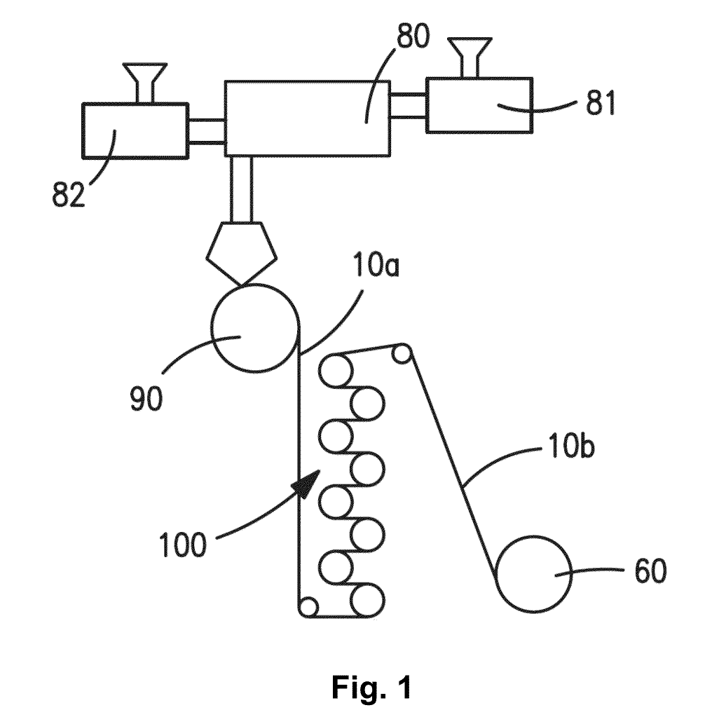

Referring to FIG. 1, for instance, one embodiment of a method for forming a co-extruded cast film is shown. In the particular embodiment of FIG. 1, the raw materials for the skin layer (not shown) are supplied to a first extruder 81 and the raw material for the core layer (not shown) are supplied to a second extruder 82. The extruders feed the compounded materials to a die 80 that casts the layers onto a casting roll 90 to form a two-layered precursor film 10a. The casting roll 90 may optionally be provided with embossing elements to impart a pattern to the film. Typically, the casting roll 90 is kept at temperature sufficient to solidify and quench the sheet 10a as it is formed, such as from about 20 to 60° C. If desired, a vacuum box may be positioned adjacent to the casting roll 90 to help keep the precursor film 10a close to the surface of the roll 90. Additionally, air knives or electrostatic pinners may help force the precursor film 10a against the surface of the casting roll 90 as it moves around a spinning roll. An air knife is a device known in the art that focuses a stream of air at a very high flow rate to pin the edges of the film.

Once cast, the film 10a may then be optionally oriented in one or more directions to further improve film uniformity and reduce thickness. Orientation may also form micropores in a film containing a filler, thus providing breathability to the film. For example, the film may be immediately reheated to a temperature below the melting point of one or more polymers in the film, but high enough to enable the composition to be drawn or stretched. In the case of sequential orientation, the “softened” film is drawn by rolls rotating at different speeds of rotation such that the sheet is stretched to the desired draw ratio in the longitudinal direction (machine direction). This “uniaxially” oriented film may then be laminated to a fibrous web. In addition, the uniaxially oriented film may also be oriented in the cross-machine direction to form a “biaxially oriented” film. For example, the film may be clamped at its lateral edges by chain clips and conveyed into a tenter oven. In the tenter oven, the film may be reheated and drawn in the cross-machine direction to the desired draw ratio by chain clips diverged in their forward travel.

Referring again to FIG. 1, for instance, one method for forming a uniaxially oriented film is shown. As illustrated, the precursor film 10a is directed to a film-orientation unit 100 or machine direction orienter (“MDO”), such as commercially available from Marshall and Willams, Co. of Providence, R.I. The MDO has a plurality of stretching rolls (such as from 5 to 8) which progressively stretch and thin the film in the machine direction, which is the direction of travel of the film through the process as shown in FIG. 1. While the MDO 100 is illustrated with eight rolls, it should be understood that the number of rolls may be higher or lower, depending on the level of stretch that is desired and the degrees of stretching between each roll. The film may be stretched in either single or multiple discrete stretching operations. It should be noted that some of the rolls in an MDO apparatus may not be operating at progressively higher speeds. If desired, some of the rolls of the MDO 100 may act as preheat rolls. If present, these first few rolls heat the film 10a above room temperature (e.g., to 125° F.). The progressively faster speeds of adjacent rolls in the MDO act to stretch the film 10a. The rate at which the stretch rolls rotate determines the amount of stretch in the film and final film weight.

The resulting film 10b may then be wound and stored on a take-up roll 60. While not shown here, various additional potential processing and/or finishing steps known in the art, such as slitting, treating, aperturing, printing graphics, or lamination of the film with other layers (e.g., nonwoven web materials), may be performed without departing from the spirit and scope of the invention.

### III. Articles

The water-sensitive biodegradable film of the present invention may be used in a wide variety of applications. For example, as indicated above, the film may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins, pantiliners, etc.), swim wear, baby wipes, and so forth; medical absorbent articles, such as garments, fenestration materials, underpads, bedpads, bandages, absorbent drapes, and medical wipes; food service wipers; clothing articles; and so forth. Several examples of such absorbent articles are described in U.S. Pat. Nos. 5,649,916 to DiPalma, et al.; 6,110,158 to Kielpikowski; 6,663,611 to Blaney, et al., which are incorporated herein in their entirety by reference thereto for all purposes. Still other suitable articles are described in U.S. Patent Application Publication No. 2004/0060112 A1 to Fell et al., as well as U.S. Pat. Nos. 4,886,512 to Damico et al.; 5,558,659 to Sherrod et al.; 6,888,044 to Fell et al.; and 6,511,465 to Freiburger et al., all of which are incorporated herein in their entirety by reference thereto for all purposes. Materials and processes suitable for forming such absorbent articles are well known to those skilled in the art.

The films of the present invention are particularly useful as backsheets for disposable absorbent articles, and in particular for flushable feminine pads, pantiliners, and interlabial pads. In such embodiments, the film is positioned so that the water-barrier skin layer faces the body of the user so that it can limit the rate of disintegration and degradation upon exposure to bodily fluids (e.g., urine, menses, etc.). Nevertheless, the presence of the water-dispersible core layer allows the film to be readily disintegrated after it is flushed.

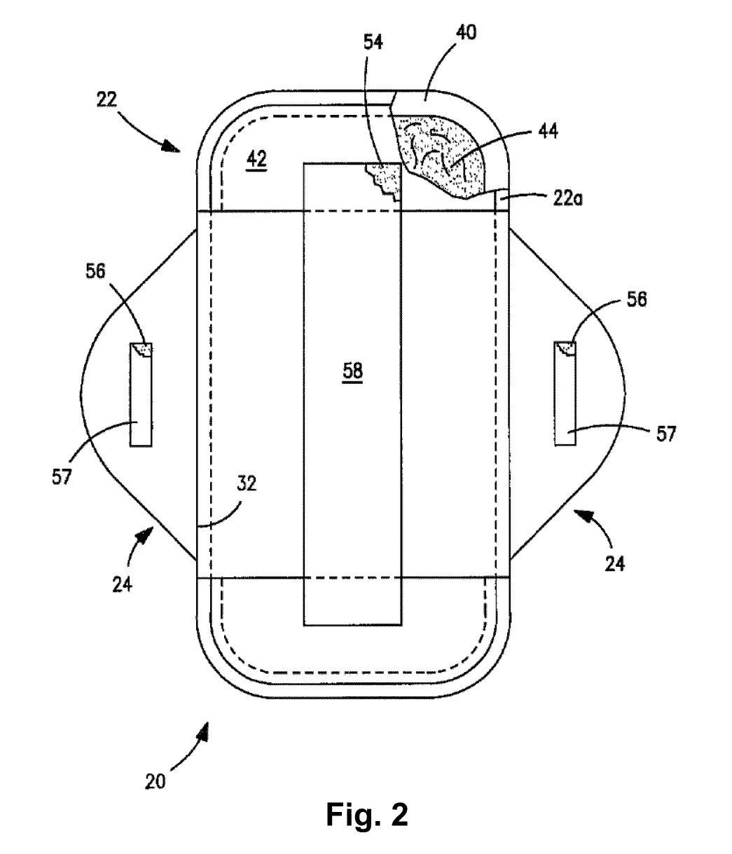

In this regard, one particular embodiment of a sanitary napkin that may employ the film of the present invention will now be described in more detail. For purposes of illustration only, an absorbent article 20 is shown in FIG. 2 as a sanitary napkin for feminine hygiene. In the illustrated embodiment, the absorbent article 20 includes a main body portion 22 containing a topsheet 40, an outer cover or backsheet 42, an absorbent core 44 positioned between the backsheet 42 and the topsheet 40, and a pair of flaps 24 extending from each longitudinal side 22a of the main body portion 22. The topsheet 40 defines a body-facing surface of the absorbent article 20. The absorbent core 44 is positioned inward from the outer periphery of the absorbent article 20 and includes a body-facing side positioned adjacent the topsheet 40 and a garment-facing surface positioned adjacent the backsheet 42.

The backsheet 42 is generally liquid-impermeable and designed to face the inner surface, i.e., the crotch portion of an undergarment (not shown). In one particular embodiment, the film of the present invention is used to form the backsheet 42 so that the water-barrier layer faces the body-facing surface and the water-dispersible layer faces the garment-facing surface. The backsheet 42 may permit a passage of air or vapor out of the absorbent article 20, while still blocking the passage of liquids.

The topsheet 40 is generally designed to contact the body of the user and is liquid-permeable. The topsheet 40 may surround the absorbent core 44 so that it completely encases the absorbent article 20. Alternatively, the topsheet 40 and the backsheet 42 may extend beyond the absorbent core 44 and be peripherally joined together, either entirely or partially, using known techniques. Typically, the topsheet 40 and the backsheet 42 are joined by adhesive bonding, ultrasonic bonding, or any other suitable joining method known in the art. The topsheet 40 is sanitary, clean in appearance, and somewhat opaque to hide bodily discharges collected in and absorbed by the absorbent core 44. The topsheet 40 further exhibits good strike-through and rewet characteristics permitting bodily discharges to rapidly penetrate through the topsheet 40 to the absorbent core 44, but not allow the body fluid to flow back through the topsheet 40 to the skin of the wearer. For example, some suitable materials that may be used for the topsheet 40 include nonwoven materials, perforated thermoplastic films, or combinations thereof. A nonwoven fabric made from polyester, polyethylene, polypropylene, bicomponent, nylon, rayon, or like fibers may be utilized. For instance, a white uniform spunbond material is particularly desirable because the color exhibits good masking properties to hide menses that has passed through it. U.S. Pat. No. 4,801,494 to Datta, et al. and U.S. Pat. No. 4,908,026 to Sukiennik, et al. teach various other cover materials that may be used in the present invention.

The topsheet 40 may also contain a plurality of apertures (not shown) formed therethrough to permit body fluid to pass more readily into the absorbent core 44. The apertures may be randomly or uniformly arranged throughout the topsheet 40, or they may be located only in the narrow longitudinal band or strip arranged along the longitudinal axis X-X of the absorbent article 20. The apertures permit rapid penetration of body fluid down into the absorbent core 44. The size, shape, diameter and number of apertures may be varied to suit one's particular needs.

The absorbent article 20 also contains an absorbent core 44 positioned between the topsheet 40 and the backsheet 42. The absorbent core 44 may be formed from a single absorbent member or a composite containing separate and distinct absorbent members. It should be understood, however, that any number of absorbent members may be utilized in the present invention. For example, in one embodiment, the absorbent core 44 may contain an intake member (not shown) positioned between the topsheet 40 and a transfer delay member (not shown). The intake member may be made of a material that is capable of rapidly transferring, in the z-direction, body fluid that is delivered to the topsheet 40. The intake member may generally have any shape and/or size desired. In one embodiment, the intake member has a rectangular shape, with a length equal to or less than the overall length of the absorbent article 20, and a width less than the width of the absorbent article 20. For example, a length of between about 150 mm to about 300 mm and a width of between about 10 mm to about 60 mm may be utilized.

Any of a variety of different materials may be used for the intake member to accomplish the above-mentioned functions. The material may be synthetic, cellulosic, or a combination of synthetic and cellulosic materials. For example, airlaid cellulosic tissues may be suitable for use in the intake member. The airlaid cellulosic tissue may have a basis weight ranging from about 10 grams per square meter (gsm) to about 300 gsm, and in some embodiments, between about 100 gsm to about 250 gsm. In one embodiment, the airlaid cellulosic tissue has a basis weight of about 200 gsm. The airlaid tissue may be formed from hardwood and/or softwood fibers. The airlaid tissue has a fine pore structure and provides an excellent wicking capacity, especially for menses.

If desired, a transfer delay member (not shown) may be positioned vertically below the intake member. The transfer delay member may contain a material that is less hydrophilic than the other absorbent members, and may generally be characterized as being substantially hydrophobic. For example, the transfer delay member may be a nonwoven fibrous web composed of a relatively hydrophobic material, such as polypropylene, polyethylene, polyester or the like, and also may be composed of a blend of such materials. One example of a material suitable for the transfer delay member is a spunbond web composed of polypropylene, multi-lobal fibers. Further examples of suitable transfer delay member materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay member are described in U.S. Pat. No. 4,798,603 to Meyer, et al. and U.S. Pat. No. 5,248,309 to Serbiak, et al., which are incorporated herein in their entirety by reference thereto for all purposes. To adjust the performance of the invention, the transfer delay member may also be treated with a selected amount of surfactant to increase its initial wettability.

The transfer delay member may generally have any size, such as a length of about 150 mm to about 300 mm. Typically, the length of the transfer delay member is approximately equal to the length of the absorbent article 20. The transfer delay member may also be equal in width to the intake member, but is typically wider. For example, the width of the transfer delay member may be from between about 50 mm to about 75 mm, and particularly about 48 mm. The transfer delay member typically has a basis weight less than that of the other absorbent members. For example, the basis weight of the transfer delay member is typically less than about 150 grams per square meter (gsm), and in some embodiments, between about 10 gsm to about 100 gsm. In one particular embodiment, the transfer delay member is formed from a spunbonded web having a basis weight of about 30 gsm.

Besides the above-mentioned members, the absorbent core 44 may also include a composite absorbent member (not shown), such as a coform material. In this instance, fluids may be wicked from the transfer delay member into the composite absorbent member. The composite absorbent member may be formed separately from the intake member and/or transfer delay member, or may be formed simultaneously therewith. In one embodiment, for example, the composite absorbent member may be formed on the transfer delay member or intake member, which acts a carrier during the coform process described above.

The absorbent article 20 also typically contains an adhesive for securing to an undergarment. An adhesive may be provided at any location of the absorbent article 20, such as on the lower surface of the backsheet 42. In this particular embodiment, the backsheet 42 carries a longitudinally central strip of garment adhesive 54 covered before use by a peelable release liner 58, which may optionally be formed from the film of the present invention. Each of the flaps 24 may also contain an adhesive 56 positioned adjacent to the distal edge 34 of the flap 24. A peelable release liner 57, which may also be formed in accordance with the present invention, may cover the adhesive 56 before use. Thus, when a user of the sanitary absorbent article 20 wishes to expose the adhesives 54 and 56 and secure the absorbent article 20 to the underside of an undergarment, the user simply peels away the liners 57 and 58 and disposed them in a water-based disposal system (e.g., in a toilet).

Of course, the film of the present invention may also be used in applications other than absorbent articles. For example, the film may be employed as an individual wrap, packaging pouch, or bag for the disposal of a variety of articles, such as food products, absorbent articles, etc. Various suitable pouch, wrap, or bag configurations for absorbent articles are disclosed, for instance, in U.S. Pat. Nos. 6,716,203 to Sorebo, et al. and 6,380,445 to Moder, et al., as well as U.S. Patent Application Publication No. 2003/0116462 to Sorebo, et al., all of which are incorporated herein in their entirety by reference thereto for all purposes.

The present invention may be better understood with reference to the following examples.

### Test Methods

### Tensile Properties:

Films were tested for tensile properties using ASTM D638-08 Standard Test Method for Tensile Properties of Plastics. A constant-rate-of-extension type of tensile tester was employed. The tensile testing system was a Sintech 1/D tensile tester, which is available from MTS Systems Corp. The tensile tester was equipped with TESTWORKS 4.08B software from MTS Systems Corp. to support the testing. An appropriate load cell was selected so that the tested value fell within the range of 10-90% of the full scale load.

Five samples were tested for each film in both the machine direction (MD) and the cross direction (CD). After conditioning for 24 hours at 70° F. and at 50% humidity, the film samples were cut into dog bone shapes with a center width of 3.0 mm before testing. The samples were held between grips on the Sintech 1/D tensile tester having a front and back face measuring 25.4 millimeters×76 millimeters. The grip faces were rubberized, and the longer dimension of the grip was perpendicular to the direction of pull. The grip pressure was pneumatically maintained at a pressure of 40 pounds per square inch. The tensile test was run using a gauge length of 18.0 millimeters and a break sensitivity of 40%. Five samples were tested by applying the test load along the machine-direction and five samples were tested by applying the test load along the cross direction. During the test, samples were stretched at a crosshead speed of about 127 millimeters per minute until breakage occurred. The computer program (Test Works 4) was used to collect data during testing and to generate a stress versus strain curve from which a number of properties were determined, including modulus, peak stress, elongation, and toughness.

### Examples 1-8

### Materials

1. Polypropylene carbonate (PPC) available as MXJJ-001 from Inner Mongolia Mengxi High-Tech Group Co., Ltd., Wuhai, Inner Mongolia, China, the brand is Melicsea. The grade MXJJ-001 with a melt flow of 3.6 g/10 minutes at 150° C., was used as an example of polyalkylene carbonate.

2. A biodegradable aliphatic-aromatic copolyester, Ecoflex® FBX 7011 (BASF Corp.) was used as an example of a biodegradable polyester with a melt flow of 4.2 g/10 minutes at 190° C.

3. Polyvinyl alcohol used was Elvanol® 51-05, a granular polymer having a degree of hydrolysis of 87.0-89.0 mole % and manufactured by DuPont.

4. Glycerin was supplied by Cognis corporation (Cincinnati, Ohio).

5. Omyacarb® 2SST calcium carbonate was manufactured by Omya, Alpharetta, Ga.

### Examples 1-4

Examples 1 to 4 were dry blends of copolyester (Ecoflex) with the PPC at weight ratios of 80:20, 60:40, 40:60 and 20:80, respectively, formed via a cast film process. The polymer blends were then processed on a cast film line having a single screw extruder with 25:1 L/D fitted with a HAAKE 6″ cast film die. In Examples 1 to 4, the EcoflexlPPC resins were dry blended at given ratios and flood fed into cast film extruder.

### Examples 5-8

Examples 5 to 8 were melt blends of the copolyester and PPC made on a Thermo Prism 16 mm twin screw extruder at weight ratios of 80:20, 60:40, 40:60 and 20:80, respectively. The blends were cooled and pelletized.

The melt blending conditions of Examples 5-8 are listed in Table 1.

TABLE 1
Processing Conditions for Melt Blending of PPC and Copolyester

| | | Set points | | | | | | | | | | | |
|---|---|---|---|---|---|---|---|---|---|---|---|---|---|
| | | 130 | 140 | 150 | 150 | 150 | 150 | 160 | 160 | 160 | 160 | | |
| | Speed | T-1 | T-2 | T-3 | T-4 | T-5 | T-6 | T-7 | T-8 | T-9 | T-10 | TQ | Pressure |
| MATERIAL | (RPM) | (° C.) | (° C.) | (° C.) | (° C.) | (° C.) | (° C.) | (° C.) | (° C.) | (° C.) | (° C.) | (%) | (bar) |
| Ecoflex/PPC at 80:20 | 140 | 130 | 140 | 150 | 150 | 150 | 150 | 160 | 160 | 160 | 160 | 87-94 | 19 |
| Ecoflex/PPC at 60:40 | 140 | 130 | 140 | 150 | 150 | 150 | 150 | 160 | 160 | 160 | 160 | 70-74 | 18 |
| Ecoflex/PPC at 40:60 | 140 | 130 | 140 | 150 | 150 | 150 | 150 | 160 | 160 | 160 | 160 | 70-74 | 16 |
| Ecoflex/PPC at 20:80 | 140 | 130 | 140 | 150 | 150 | 150 | 150 | 160 | 160 | 160 | 160 | 74-82 | 15 |

Example 5 (copolyester Ecoflex®/PPC at 80:20 ratio) showed a smooth surface on strands that were white in color. The strands were strong when pulled upon. Materials were dry blended in solid state (1000 grams total) and fed into Thermo Prism extruder at 3 pounds/hour.

Example 6 (copolyester Ecoflex®/PPC at 60:40 ratio) showed a smooth surface on strands that were white in color. Strands have good strength to them when pulled upon. Materials were dry blended in solid state (1000 grams total) and fed into Thermo Prism extruder at 3 pounds/hour.

Example 7 (copolyester Ecoflex®/PPC at 40:60 ratio) showed a smooth surface on strands that were white in color. Strands have good strength to them when pulled upon. Noticed gargling sound coming from the throat of the extruder, could be build-up of moisture. No moisture build-up visible to the eyes. Materials were dry blended in solid state (1000 grams total) and fed into Thermo Prism extruder at 3 pounds/hour.

Example 8 (copolyester Ecoflex®/PPC at 20:80 ratio) showed a smooth surface on strands that were white in color. Strands have good strength to them when pulled upon. Noticed gargling sound coming from the throat of the extruder, could be build-up of moisture. No moisture build-up visible to the eyes. Materials dry blended in solid state (1000 grams total) and fed into Thermo Prism extruder at 3 pounds/hour.

### Cast Film Process

The cast film conditions are listed in Table 2. The same process conditions were used for all 8 examples. However, due to composition difference, the resultant die pressures and torques are observed and summarized in Table 2. As a comparison, two comparative examples (1 and 2) were formed from pure PPC and pure Ecoflex, respectively.

TABLE 2
Cast Film Process Conditions for PPC and Copolyester Blends

| | | Temperatures | | | | | | |
|---|---|---|---|---|---|---|---|---|
| | | 170 | 175 | 178 | 180 | | | |
| | Speed | T-1 | T-2 | T-3 | Die | Tm | Die | Torque |
| MATERIAL | (RPM) | (° C.) | (° C.) | (° C.) | (° C.) | (° C.) | (Bar) | (N · m) |
| Example 1: Copolyester/PPC | 50 | 170 | 175 | 178 | 180 | 194 | 12 | 9 |
| 80:20 (Dry Blend: DB) | | | | | | | | |
| Example 2: Copolyester/PPC | 50 | 170 | 175 | 178 | 180 | 194 | 10 | 8 |
| 60:40 (DB) | | | | | | | | |
| Example 3: Copolyester/PPC | 50 | 170 | 175 | 178 | 180 | 194 | 9 | 7 |
| 40:60 (DB) | | | | | | | | |
| Example 4: Copolyester/PPC | 50 | 170 | 175 | 178 | 180 | 194 | 7 | 5 |
| 20:80 (DB) | | | | | | | | |
| Example 5: Copolyester/PPC | 50 | 170 | 175 | 178 | 180 | 194 | 14 | 12 |
| 80:20 (Melt Blend: MB) | | | | | | | | |
| Example 6: Copolyester/PPC | 50 | 170 | 175 | 178 | 180 | 194 | 7 | 10 |
| 60:40 (MB) | | | | | | | | |
| Example 7: Copolyester/PPC | 50 | 170 | 175 | 178 | 180 | 194 | 6 | 10 |
| 40:60 (MB) | | | | | | | | |
| Example 8: Copolyester/PPC | 50 | 170 | 175 | 178 | 180 | 194 | 5 | 8 |
| 20:80 (MB) | | | | | | | | |

Example 1 (copolyester/PPC DB at 80:20 ratio) formed a film having a smooth surface, translucent and tough.

Example 2 (copolyester/PPC DB at 60:40 ratio) formed a film having a smooth surface, semi soft to the touch, noisy when ruffled, translucent. No blocking or adhesion was observed when rolled upon itself.

Example 3 (copolyester/PPC DB at 40:60 ratio) formed a film having a smooth shiny surface, semi soft to the touch, noisy when ruffled, translucent. No blocking or adhesion was observed when rolled upon itself.

Example 4 (copolyester/PPC DB at 20:80 ratio) formed a film having a smooth shiny surface, noisy when ruffled, translucent. Slight blocking or adhesion was observed when rolled upon itself.

Example 5 (copolyester/PPC (MB) at 80:20 ratio) formed a film having a smooth surface, soft to the touch, translucent and tough. Slight noise was observed when ruffled. Feels like LDPE.

Example 6 (copolyester/PPC (MB) at 60:40 ratio) formed a film having a smooth surface, soft to the touch, translucent and tough. Slight noise was observed when ruffled.

Example 7 (copolyester/PPC (MB) at 40:60 ratio) formed a film having a smooth shiny surface, semi soft to the touch, noisy when ruffled, translucent. No blocking or adhesion was observed when rolled upon itself.

Example 8 (copolyester/PPC (MB) at 20:80 ratio) formed a film having a smooth shiny surface, noisy when ruffled, translucent. Slight blocking or adhesion was observed when rolled upon itself.

Comparative Example 1 Melicsea PPC (100%) formed a film that was soft to the touch and transparent. The film would block or adhere to itself when rolled up shortly after making the film.

Comparative Example 2 copolyester Ecoflex® (100%) formed a film that was soft to the touch, transparent and tough.

### Testing Mechanical Properties of Cast Films

The films were tested for tensile properties using ASTM D638-08 Standard Test Method for Tensile Properties of Plastics, as described above. Five samples were tested for each film in both the machine direction (MD) and the cross direction (CD). A computer program Test Works 4 was used to collect data during testing and to generate a stress versus strain curve from which a number of properties were determined, including modulus, peak stress, elongation, and toughness.

After conditioning for 24 hours at 70F @ 50% humidity the film samples were cut into dog bone shapes with a center width of 3.0 mm before testing. The dog-bone film samples were held in place using grips on the Sintech device with a gauge length of 18.0 mm. The film samples were stretched at a crosshead speed of 127.0 mm/min until breakage occurred.

All these blend films have good mechanical properties, as shown in Tables 3 and 4. Due to the cast film process, MD properties were better than CD properties. Surprisingly, the dry blend films-examples 1 to 4 had apparently slight better peak stress than those of melt blend examples 5 to 8. In examples 1 to 4, the MD peak stress ranges from 26 to 38 MPa, while the strain at break in MD ranges from about 340% to about 670%. Surprisingly, the moduli in Examples 2 to 4 are higher than either pure PPC or copolyester films, this is not expected but it could be the results of the reinforcing or stiffening effects by the morphological structures.

Examples 5 to 8 had peak stress ranges from 20 to 38 MPa and strain at break from about 310 to about 700% in MD.

TABLE 3
MD Tensile Properties of PPC and Copolyester Blend Cast Films

| | | | | | | | | | | Energy |
|---|---|---|---|---|---|---|---|---|---|---|
| | | | | | | | | | | per |
| | | Peak | Peak | Strain @ | | Break | Load @ | Stress @ | Strain @ | Volume @ |
| | Thickness | load | stress | break | Modulus | stress | yield | yield | yield | break |
| Examples | (mil) | (gf) | (MPa) | (%) | (MPa) | (MPa) | (gf) | (MPa) | (%) | (J/cm\hat{ }3 |
| Example 1 | 1.06 | 313 | 38 | 669 | 400 | 38 | 92 | 11 | 3 | 139 |
| Example 2 | 1.1 | 220 | 26 | 441 | 822 | 26 | 169 | 20 | 3 | 75 |
| Example 3 | 1.15 | 282 | 32 | 339 | 871 | 32 | 197 | 22 | 3 | 65 |
| Example 4 | 1.12 | 287 | 33 | 361 | 1074 | 33 | 246 | 28 | 4 | 73 |
| Example 5 | 1.15 | 203 | 23 | 704 | 204 | 23 | 70 | 8 | 9 | 87 |
| Example 6 | 1.15 | 178 | 20 | 312 | 551 | 20 | 118 | 13 | 3 | 43 |
| Example 7 | 1.16 | 207 | 23 | 526 | 1014 | 16 | 207 | 23 | 2 | 76 |
| Example 8 | 1.09 | 321 | 38 | 346 | 1511 | 21 | 321 | 38 | 3 | 61 |
| Ecoflex (100%) | 0.975 | 397 | 52 | 200 | 159 | 52 | 52 | 7 | 6 | 71 |
| Melicsea PPC (100%) | 1.1 | 175 | 20 | 587 | 584 | 20 | 161 | 18 | 5 | 74 |

TABLE 4
CD Tensile Properties of PPC and Copolyester Blend Cast Films

| | | | | | | | | | | Energy |
|---|---|---|---|---|---|---|---|---|---|---|
| | | | | | | | | | | per |
| | | Peak | Peak | Strain @ | | Break | Load @ | Stress @ | Strain @ | Volume @ |
| | Thickness | load | stress | break | Modulus | stress | yield | yield | yield | break |
| Examples | (mil) | (gf) | (MPa) | (%) | (MPa) | (MPa) | (gf) | (MPa) | (%) | (J/cm\hat{ }3 |
| Example 1 | 1.1 | 165 | 19 | 571 | 207 | 19 | 71 | 8 | 6 | 72 |
| Example 2 | 1.1 | 73 | 9 | 40 | 345 | 8 | 70 | 8 | 3 | 3 |
| Example 3 | 1.05 | 103 | 13 | 205 | 754 | 12 | 101 | 12 | 2 | 23 |
| Example 4 | 1.13 | 179 | 20 | 8 | 771 | 15 | 179 | 20 | 3 | 0.991 |
| Example 5 | 1.1 | 108 | 13 | 679 | 156 | 13 | 51 | 6 | 7 | 55 |
| Example 6 | 1.05 | 35 | 4 | 79 | 137 | 4 | 32 | 4 | 3 | 3 |
| Example 7 | 1.15 | 39 | 4 | 2 | 231 | 4 | 39 | 4 | 2 | 0.043 |
| Example 8 | 1 | 121 | 16 | 1 | 1066 | 16 | 121 | 16 | 1 | 0.112 |
| Ecoflex (100%) | 1 | 395 | 51 | 1029 | 160 | 51 | 61 | 8 | 9 | 182 |
| Melicsea PPC | 1.2 | 174 | 19 | 569 | 744 | 18 | 170 | 18 | 5 | 69 |
| (100%) | | | | | | | | | | |

### Morphology of the Polymer Blends

All films were prepared identically. The direction of cut was made across the CD, which was determined from the visible striations in the films. Two pieces were cut out from different locations in the film. These were chilled for 1 minute in liquid nitrogen vapor (not immersed in a liquid nitrogen bath) to stiffen followed by rapid cutting using a chilled Teflon-coated surgical razor. The sections were then mounted on aluminum SEM stubs with conductive carbon tape. Immediately, the samples were placed in a plasma processing unit (Emitech Model K1050X) and lightly oxygen plasma etched for 3 min. with O₂ plasma. With this regimen the sample temperature should remain well below 90° C. The plasma etch enhances the phase structure and provides improved contrast for secondary electron SEM imaging. Immediately after plasma processing was complete the samples were sputter coated 2-minutes with gold using a Denton Desk V sputter coater. The samples were then imaged in a JEOL 6490LV SEM operating with 7 kV electron beam.

For each sample, the pair of sections were scanned to assess the typical morphology prior to imaging. Images at 3000×, 6000×, and 15,000× were then taken. These images show the respective phase separation and structure representative for that code.

The SEM of Example 1 is shown in FIG. 3. The dispersed phase is PPC, present in the film as finely dispersed ellipsoidal structures ranges from submicron to 2 up to 2 to 3 microns.

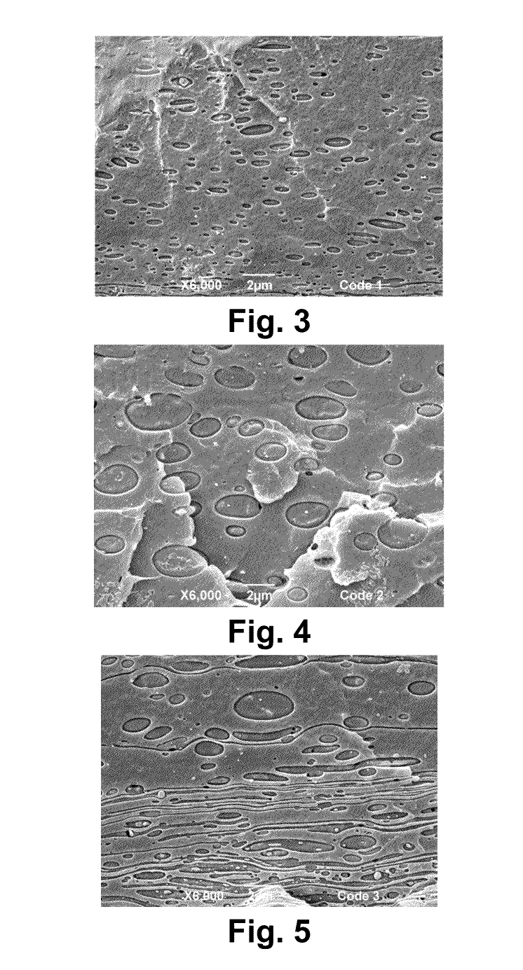

The SEM image of Example 5 is shown in FIG. 4. Example 5 has the same composition as Example 2 but was melt blended. The sizes of dispersed phase in FIG. 4 are significantly larger than FIG. 3, with mostly large dispersed structures up to 4 to 5 microns. This is not expected, typically, melt blending produces a finer dispersion of the dispersed phase than the dry blend process due to the high shear deformation and mixing in melt blending process. This morphological change showed that the morphology of PPC and copolyester is more kinetically controlled during melt processing, the dispersed phase coalesced during melt extrusion to form larger structures.

The SEM of Example 2 is shown in FIG. 5. This dry blend at 60% of copolyester and 40% PPC had two types of dispersed phase, laminar type near surface and droplet (oval) type structure near the center part of the film.

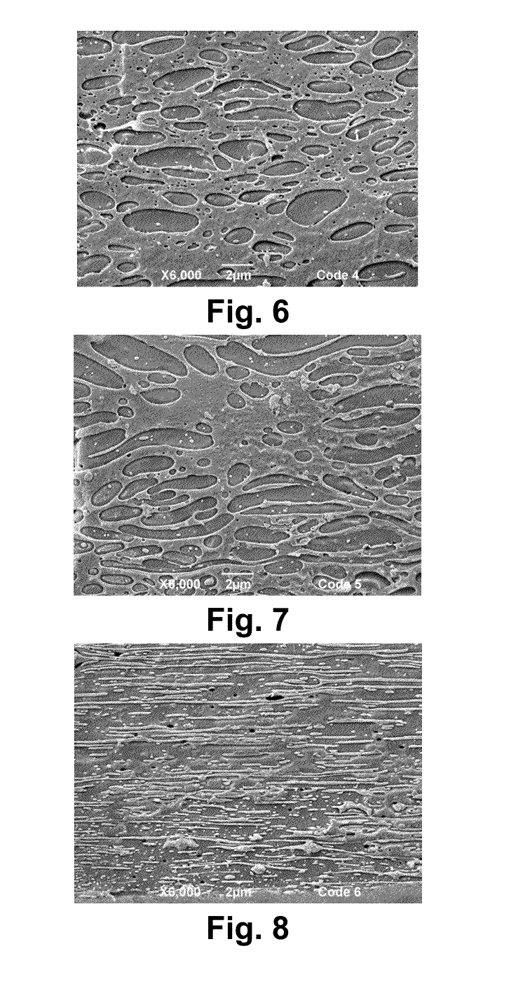

The SEM image of Example 6 at the same composition of Example 2 but melt blended is shown in FIG. 6. It is quite different, in contract to FIG. 5, laminar type dispersion is absent. There are large dispersions of PPC and nano-sized sipersion of PPC co-existing. Again, this showed the effects of processing method on the morphology of the PPC and copolyester blends.

The SEM image of Example 7 (melt blended copolyester/PPC at 40160) is shown in FIG. 7. This melt blend with a majority of PPG has, surprisingly, a dispersed phase of PPC which is expected to form a continuous phase based on melt volumetric considerations. Therefore, there is an unexpected phase inversion. This could result from a combination of rheological difference of the two polymer phases and interfacial tensions.

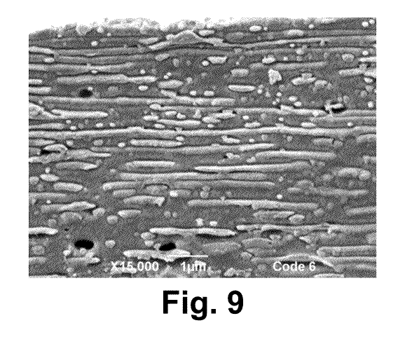

The SEM image of Example 8 with melt blended 20% copolyester and 80% PPC (by weight) is shown in FIG. 8. The blend film has an expected normal phase structure, i.e. the PPC is the continuous phase and copolyester is the dispersed phase. However, at 20% level of copolyester, it formed a totally different morphology versus the 20% PPC blend film (Example 2 in FIG. 4). There are two types of structures formed from the copolyester, one is a fine laminar type and the other a finely dispersed droplet type in almost nano-range dispersion. A further amplified SEM image is shown in FIG. 9.

### Example 9

A plasticized polyvinyl alcohol was prepared follows. The composition of a water-dispersible composition consists of 71 parts of polyvinyl alcohol (Elvanol 51-05, a granular polymer having a degree of hydrolysis of 87.0-89.0 mole %, and manufactured by DuPont), 14% plasticizer (glycerin, supplied by Cognis corporation, Cincinnati, Ohio), and 15% calcium carbonate (Omyacarb® 2SST calcium carbonate from Omya, Alpharetta, Ga.). Polyvinyl alcohol was fed to feed hopper of a ZSK-30 (Werner Pfleiderer Corporation, Ramsey, N.J.) co-rotating, twin-screw extruder (having 14 barrels and a screw length of 1328 mm) using a gravimetric K-Tron feeders (K-Tron America, Pitman, N.J.). Calcium carbonate was fed using a ZSB-25 side stuffer (Werner Pfleiderer Corporation, Ramsey, N.J.) located between Barrels #6 and #7. Glycerine was introduced into Barrel #2 using an injector, glycerine was pumped by a 3-head piston pump (made by Eldex), The extruder had seven heating zones, the temperatures for the seven heating zones from Zone 1 to Zone 7 (from feed hopper to die) were 90° C., 150° C., 185° C., 190° C., 180° C., 170° C., and 165° C., respectively. The screw speed was 150 rpm. The melt pressure was 60-100 psi. The torque was 64-70%, The strands were cooled on a fan-cooled conveyor cooling belt (Minarik Electric Company, Glendale, Calif.). A pelletizer (Conair, Bay City, Mich.) was used to cut the strand to produce water-dispersible polymer pellets, which were then collected and sealed in a bag.

### Example 10

Two polymer blends were prepared separately on a twin screw extruder. Each of the blends was then extruded into a film:

1, A melt blend of a water dispersible polyvinyl alcohol polymer (PVOH), glycerin, and CaCO₃ at a weight ratio of 71:14:15; and

2. A melt blend of Ecoflex (BASF) and PPC (Melicsea MXJJ-001) at a weight ratio of 60:40.

These polymer blends where processed on a HAAKE cast film line which consisted of a single screw extruder (1.0=25:1) attached with a HAAKE 6″ cast film die to produce two films according to the film casting processing conditions set forth in Table 5.

TABLE 5
Film casting processing conditions

| | | | | | Screw | | | Melt |
|---|---|---|---|---|---|---|---|---|
| | Z-1 | Z-2 | Z-3 | Die | speed | Torque | Pressure | Temp |
| PVOH/glycerin/CaCO₃ | 160 | 165 | 170 | 175 | 60 | 21 | 38 | 193 |
| Ecoflex/PPC | 170 | 175 | 178 | 180 | 50 | 8 | 10 | 194 |

The resulting films were then pressed together using a 15-ton hydraulic Carver press. The press has two platens and they both where set at 250° F. with a dwell time of 1.5 minutes under 14,500 lbs. force.

The films were then conditioned at 75° F. at 50% relative humidity overnight. After conditioning overnight the films were taken out of conditioning room and mechanical testing was performed using a Synergie 200 tensile frame. The load cell was 100 Newton (−22 lbs) and we are using Test works 4 program. This program documents Peak Load (gf), Peak Stress (MPa), Strain at Break (%), Modulus (MPa), Break Stress (MPa), Load at Yield (gf), Stress at Yield (MPa), Strain at Yield (%) and Energy Per Volume at Break (J/cm³), with results set forth in Table 6. For comparison (comparative examples 1 and 2), samples of the two films alone, without lamination together, were also tested.

TABLE 6
Pressed Film Tensile Properties
MD Tensile

| | | | | | | | | | | Energy |
|---|---|---|---|---|---|---|---|---|---|---|
| | | | | | | | | | | per |
| | | | Peak | Strain @ | | Break | Load @ | Stress @ | Strain @ | Volume @ |
| | Thickness | Peak | stress | break | Modulus | stress | yield | yield | yield | break |
| Sample | (mil) | load (gf) | (MPa) | (%) | (MPa) | (MPa) | (gf) | (MPa) | (%) | (J/cm³) |
| Comparative | 4.2 | 941 | 29 | 328 | 106 | 29 | 343 | 11 | 24 | 60 |
| example #1 | | | | | | | | | | |
| (PVOH/glycerin/ | | | | | | | | | | |
| CaCO₃) | | | | | | | | | | |
| Comparative | 1.1 | 220 | 26 | 441 | 822 | 26 | 169 | 20 | 3 | 75 |
| example #2 | | | | | | | | | | |
| (Ecoflex/PPC) | | | | | | | | | | |
| Example #1 | 2.4 | 461 | 25 | 166 | 281 | 25 | 223 | 12 | 7 | 31 |
| (PVOH/glycerin/ | | | | | | | | | | |
| CaCO₃)- | | | | | | | | | | |
| (Ecoflex/PPC) | | | | | | | | | | |
| pressed film | | | | | | | | | | |

CD Tensile

| | | | | | | | | | | Energy |
|---|---|---|---|---|---|---|---|---|---|---|
| | | | | | | | | | | per |
| | | | Peak | Strain @ | | Break | Load @ | Stress @ | Strain @ | Volume @ |
| | Thickness | Peak | stress | break | Modulus | stress | yield | yield | yield | break |
| Sample | (mil) | load (gf) | (MPa) | (%) | (MPa) | (MPa) | (gf) | (MPa) | (%) | (J/cm\hat{ }3) |
| Comparative | 4.2 | 769 | 24 | 335 | 89 | 24 | 314 | 10 | 28 | 52 |
| example #1 | | | | | | | | | | |
| (PVOH/glycerin/ | | | | | | | | | | |
| CaCO₃) | | | | | | | | | | |
| Comparative | 1.1 | 73 | 9 | 40 | 345 | 8 | 70 | 8 | 3 | 3 |
| example #2 | | | | | | | | | | |
| (Ecoflex/PPC) | | | | | | | | | | |
| Example #1 | 2.5 | 352 | 16 | 147 | 233 | 16 | 247 | 11 | 10 | 20 |
| (PVOH/glycerin/ | | | | | | | | | | |
| CaCO₃)- | | | | | | | | | | |
| (Ecoflex/PPC) | | | | | | | | | | |
| pressed film | | | | | | | | | | |

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A biodegradable and flushable film having a thickness of about 50 micrometers or less, the film comprising:
a water-dispersible core layer that comprises a water-soluble polymer; and
a water-barrier skin layer positioned adjacent to the water-dispersible core layer, the water-dispersible core layer constituting from about 50 wt. % to about 99 wt. % of the film and the water-barrier skin layer constituting from about 1 wt. % to about 50 wt. % of the film, wherein biodegradable polymers constitute from about 80 wt. % to 100 wt. % of the polymer content of the water-barrier skin layer, wherein from about 10 wt. % to about 90 wt. % of the biodegradable polymers are polyalkylene carbonate and from about 10 wt. % to about 90 wt. % of the biodegradable polymers are biodegradable polyesters.

2. The film of claim 1, wherein the biodegradable polymers of the water-barrier skin layer comprise about 60 wt. % or less of the polyalkylene carbonate such that the polyalkylene carbonate defines dispersed domains within a continuous phase formed by the biodegradable polyester.

3. The film of claim 1, wherein the biodegradable polymers of the water-barrier skin layer comprise about 65 wt. % or more of the polyalkylene carbonate such that the polyalkylene carbonate defines a continuous phase with dispersed domains of the biodegradable polyester.

4. The film of claim 1, wherein the polyalkylene carbonate is a polypropylene carbonate.

5. The film of claim 1, wherein the polyalkylene carbonate is a polyethylene carbonate.

6. The film of claim 1, wherein the polyalkylene carbonate is a homopolymer.

7. The film of claim 1, wherein the water-barrier skin layer further comprises a compatibilizer that has a component compatible with the polyalkylene carbonate and another component compatible with the polyester.

8. The film of claim 7, wherein the compatibilizer comprises a graft co-polymer.

9. The film of claim 1, wherein the water-barrier skin layer is free of a compatibilizer, plasticizer, or both.

10. The film of claim 1, wherein the biodegradable polyesters includes an aliphatic polyester, an aliphatic-aromatic copolyester, or a combination thereof.

11. The film of claim 1, wherein the water-soluble polymer comprises a vinyl alcohol polymer.

12. The film of claim 1, wherein the water-dispersible layer further comprises a biodegradable polymer.

13. The film of claim 12, wherein the water-dispersible layer contains a chemically modified starch polymer and a biodegradable polyester.

14. The film of claim 13, wherein chemically modified starch polymers constitute from about 30 wt. % to about 70 wt. % of the polymer content of the water-dispersible layer and biodegradable polyesters constitute from about 10 wt. % to about 40 wt. % of the polymer content of the water-dispersible layer.

15. The film of claim 1, wherein the water-dispersible layer further contains a filler.

16. The film of claim 15, wherein the water-dispersible layer comprises a vinyl alcohol polymer, a plasticizer, and an inorganic filler.

17. The film of claim 15, wherein fillers constitute from about 1 wt. % to about 30 w. % of the water-dispersible layer.

18. The film of claim 1, wherein the film exhibits an ultimate tensile strength in the machine direction of from about 10 MPa to about 80 MPa.

19. The film of claim 1, wherein the film exhibits a Young's modulus in the machine direction of from about 50 MPa to about 1200 MPa.

20. A packaging film comprising the film of claim 1.

21. The packaging film of claim 20, wherein the packaging film forms a wrap, a pouch, or a bag.

22. A method of forming a biodegradable and flushable film having a thickness of about 50 micrometers or less, the method comprising:
co-extruding a water-dispersible core layer and a water-barrier skin layer to form the film, wherein the water-dispersible core layer comprises a water-soluble polymer, and wherein biodegradable polymers constitute from about 80 wt. % to 100 wt. % of the polymer content of the water-barrier skin layer, wherein from about 10 wt. % to about 90 wt. % of the biodegradable polymers are polyalkylene carbonate and from about 10 wt. % to about 90 wt. % of the biodegradable polymers are biodegradable polyesters.

23. The method of claim 22, wherein the water-barrier skin layer is formed from a dry blended polymeric mixture comprising the polyalkylene carbonate and the biodegradable polyesters.

24. The method of claim 22, wherein the water-barrier skin layer is formed from a melt blended polymeric mixture comprising the polyalkylene carbonate and the biodegradable polyesters.

25. An absorbent article that comprises:
a liquid permeable topsheet;
a generally liquid impermeable backsheet; and
an absorbent core positioned between the backsheet and the topsheet;
wherein the backsheet includes a biodegradable and flushable film comprising a water-dispersible core layer positioned adjacent to a water-barrier skin layer, wherein the water-dispersible core layer comprises a water-soluble polymer, and wherein biodegradable polymers constitute from about 80 wt. % to 100 wt. % of the polymer content of the water-barrier skin layer, wherein from about 10 wt. % to about 90 wt. % of the biodegradable polymers are polyalkylene carbonate and from about 10 wt. % to about 90 wt. % of the biodegradable polymers are biodegradable polyesters.

26. The absorbent article as in claim 25, wherein the absorbent article is a feminine pad, a pantiliner, or an adult incontinence pantiliner.

27. The absorbent article as in claim 25, wherein the absorbent article is a disposable absorbent article.

28. The absorbent article as in claim 27, wherein the disposable absorbent article is a flushable feminine pad, a flushable pantiliner, or a flushable interlabial pad.
